(19) United States
(12) Patent Application Publication
(10) Pub. No.: US 2024/0344014 A1
(43) Pub. Date: Oct. 17, 2024

(54) 3D SHELL MICROELECTRODE ARRAYS FOR ELECTRICALLY ACTIVE ORGANOIDS

(71) Applicant: The Johns Hopkins University, Baltimore, MD (US)

(72) Inventors: Qi HUANG, Baltimore, MD (US); Lena SMIRNOVA, Baltimore, MD (US); David H. GRACIAS, Baltimore, MD (US)

(73) Assignee: The Johns Hopkins University, Baltimore, MD (US)

(21) Appl. No.: 18/635,861

(22) Filed: Apr. 15, 2024

Related U.S. Application Data
(60) Provisional application No. 63/459,133, filed on Apr. 13, 2023.

(51) Int. Cl. C12M 1/34; C12M 3/00

(52) U.S. Cl. CPC C12M 41/46; C12M 23/48

(57) ABSTRACT

A device for electrically connecting with and holding organoids includes a substrate and a multielectrode array (MEA) attached to said substrate. The MEA includes an electrical bus attached to the substrate, and a plurality of leaflets each having a first end pivotably connected to the electrical bus while remaining portions thereof are free to move relative to the substrate while remaining connected and pivoting at each first end. Each leaflet of the plurality of leaflets includes an electrical wire embedded therein that extends from each respective leaflet into the electrical bus. The MEA has a size that is suitable to electrically connect to and hold an organoid that is between 250 μm to 2 cm while at least one leaflet of the plurality of leaflets is pivoted up away from the substrate so as to securely hold the organoid in place and form a three-dimensional electrical connection with the organoid.

### CROSS-REFERENCE TO RELATED APPLICATIONS

The present patent application claims priority benefit to U.S. Provisional Patent Application No. 63/459,133, filed on Apr. 13, 2023, the entire content of which is incorporated herein by reference. All references cited anywhere in this specification, including the Background and Detailed Description sections, are incorporated by reference as if each had been individually incorporated.

### FEDERAL FUNDING

This invention was made with government support under grant R83950501 awarded by the Environmental Protection Agency. The government has certain rights in the invention.

### BACKGROUND

### 1. Technical Field

The currently claimed embodiments of the present invention relate to microelectrode arrays (MEAs) for electrically active organoids, and more particularly to MEAs that hold electrically active organoids in place and have three-dimensional (3D) electrical connections thereto.

### 2. Discussion of Related Art

As direct research on the human brain has been practically and ethically limited and animal models have limitations of interspecies differences, in vitro models utilizing human cells have emerged as an attractive alternative approach to understanding neuronal circuitry, neurotoxicity, neurological disorders, and brain development (1-3). Notably, there has been remarkable progress in the field of human in vitro models in recent years (4,5). In particular, pluripotent stem cell-derived brain organoids, with their three-dimensional (3D) multicellular architecture, and development profile, have been shown to replicate key features of the human brain (6-11). Concurrent with the development of brain organoids is the need to develop electronic and optical infrastructure for in situ stimulation and recording of electrical activity to assess their functionality and physiological relevance.

Multielectrode arrays (MEAs) provide non-invasive and high-speed recording and network mapping of extracellular electric field potential (12-14). However, traditional in vitro MEA plates use predominantly planar electrode interfaces, initially designed for monolayer cultures, thus limiting the contact surface area with 3D organoids (3, 15). Several modifications such as spine-shaped electrodes, nanowires, and 3D nanostructures have been patterned on these MEA plates to increase the signal. For example, spine-shaped gold electrodes have been used to reduce the extracellular cleft for a better signal-to-noise ratio (16), and vertical nanowires provide recording access to the interior of the measured cell via penetration (17). Nevertheless, even in such devices, the recording contact area would still be limited to the bottom of the organoid where it is attached to the plate (16-19). Recently, several curved and folded shapes have been introduced for MEA recording, including buckled, cylindrical, and shells (20-26). For example, we have previously utilized SiO/SiO2 bilayers to create a self-folding shell for single/few cardiac cell encapsulation and measurement, but the small dimensions and rigid material composition of such single-cell devices may not be scalable and suitable for larger brain organoids (20). Kalmykov et al. have developed a cylindrical sensor array for cardiac and cortical organoids (21, 23). This methodology requires manual unrolling of the cylinder with a micromanipulator to enclose the organoids which can limit throughput and also has a limited cylindrical shape. Park et al. have demonstrated buckled mesoscale spheroid neuro-interface for brain organoid recording (24). This approach requires a pre-strained elastomeric substrate, which can limit integration with other silicon modules or microfluidic devices. Therefore, there remains a need for new and/or improved microelectrode arrays (MEAs) for electrically active organoids.

### SUMMARY

A device for electrically connecting with and holding organoids according to an embodiment of the current invention includes a substrate and a multielectrode array (MEA) attached to said substrate. The MEA includes an electrical bus attached to the substrate, and a plurality of leaflets each having a first end pivotably connected to the electrical bus while remaining portions thereof are free to move relative to the substrate while remaining connected and pivoting at each first end. Each leaflet of the plurality of leaflets includes an electrical wire embedded therein that extends from each respective leaflet into the electrical bus. The MEA has a size that is suitable to electrically connect to and hold an organoid that is between 250 μm to 2 cm while at least one leaflet of the plurality of leaflets is pivoted up away from the substrate so as to securely hold the organoid in place and form a three-dimensional electrical connection with the organoid.

A method of producing a multielectrode array (MEA) for electrically connecting with organoids according to an embodiment of the current invention includes providing a substrate; forming a pattern layer of sacrificial material on the substrate; forming a first polymer layer including a plurality of lower leaflets over corresponding portions of the sacrificial layer of material and a lower bus section in direct contact with the substrate; forming a plurality of wires over the plurality of lower leaflets such that there is at least one wire over each lower leaflet and each of the plurality of wires extends to the lower bus section; and forming a second polymer layer comprising a plurality of upper leaflets over corresponding lower leaflets and an upper bus section over the lower bus section. The plurality of wires are embedded between the first and the second polymer layers, and the upper and lower leaflets have a size and a pre-stressed portion such that they fold up to a size to electrically connect with and hold an organoid, the organoid having a maximum dimension of between 250 μm to 2 cm.

A device for electrically connecting with and holding organoids according to an embodiment of the current invention is produced according to a method according to an embodiment of the current invention.

### BRIEF DESCRIPTION OF THE DRAWINGS

Embodiments of the present invention, as well as the methods of operation and functions of the related elements of structure and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIGS. 1A-1E show a fabrication process flow and experimental images of the 3D shell MEAs according to an embodiment of the current invention. (A) Fabrication process flow of 3D shell MEAs. (B) An optical image of the fabricated shell MEAs. (C) A zoomed-in optical image of the shell electrodes in the flat state. Scale bar: 200 μm. (D) Scanning electron microscopy (SEM) image of the shell MEAs after actuation. Scale bar: 100 μm. (E) The recording electrodes on the leaflet before (left) and after (right) conductive polymer PEDOT: PSS electroplating. Scale bar: 50 μm.

FIGS. 2A-2C show electrodeposition characteristics of PEDOT: PSS. A) Plot showing the electrode impedance as a function of the conductive polymer PEDOT: PSS electroplating time. (B) Plot showing the impedance change of 6 PEDOT: PSS electrodes after 10 days of soaking in cell medium. The data indicates that the impedance does not change significantly over this time. (C) Laser scanning microscopy height profiles of the PEDOT: PSS coated gold electrodes at different electroplating times.

FIG. 3 shows the location of the reference electrode relative to the shell MEA according to an embodiment of the current invention. An optical image shows the position of the reference electrode relative to the shell electrodes in the pre-folded state. Scale bar: 1000 μm.

FIGS. 4A-4C show an example of a brain organoid model according to an embodiment of the current invention. (A) Eight-week brain organoid stained with neuronal marker MAP2 (green) and neuroprogenitor marker, Nestin (red). (B) Eight-week brain organoid stained with neuronal marker β-III-Tubulin (green) and astrocyte marker, GFAP (red). Nuclei are stained with Hoechst (blue). Scale bar: 100 μm. (C) RT-PCR showing expression of neuroprogenitor (Nestin), neuron (NeuN), astrocyte (GFAP), and oligodendrocyte (MBP) genes over the course of 10 weeks of differentiation. Data is shown as Mean±SEM, n=3. Map2—Microtubule Associated Protein 2, GFAP-Glial Fibrillary Acidic Protein, MBP-Myelin Basic Protein, NPC-Neuroprogenitors, 2 w, 4 w, 8 w, 10 w-2, 4, 8, 10 weeks of differentiation starting from NPC stage.

FIGS. 5A-5C show an example of an initial electrode configuration and brain organoid encapsulation according to an embodiment of the current invention. (A) Image of a 3D shell MEA in the pre-folded (as-fabricated) state. (B) Top view of a fluorescently labelled brain organoid captured within a 3D shell MEA. (Blue: SU8, Green: Fluo-4 labeled brain organoid) (C) Side view showing an example of self-folded leaflets not able to accurately encapsulate the brain organoid suggesting the need for optimization of leaflets and folding.

FIGS. 6A-6E show FEM simulation of the programmable folding of 3D MEAs and optical images of brain organoid encapsulation according to an embodiment of the current invention. (A) Plot depicting the simulated radius of curvature (ROC) as a function of the SU8 bilayer thickness and the top layer exposure energy. The top left image shows the size of the shell in flat state. The tip-to-tip distance from left to right is 1450 μm. (B) FEM snapshots showing organoids of different sizes (400-600 μm) fitting in tailored shell electrodes. (C) Corresponding SEM images of 3D shell electrodes with different levels of folding. The images are false-colored with blue indicating the SU8 shell and yellow indicating the electrodes. Scale bar: 100 μm. (D) Brightfield image of the organoid in a 3D shell MEA, and (E) Confocal image showing the top view (projected confocal stack) of a brain organoid (green, Fluo-4 calcium dye) with a diameter around 500 μm encapsulated in the 3D shell (blue) electrodes. Scale bar: 100 μm.

FIG. 7 shows typical time scale of the self-folding of the 3D shell MEAs according to an embodiment of the current invention. The time-lapse images show the self-folding process of the 3D shell MEAs. The folding process is slow enough to enable secure placement and capture of the organoid. Scale bar: 250 μm.

FIG. 8 shows reversibility of self-folding of the shell MEAs according to an embodiment of the current invention. The image snapshots show the reversible folding of the 3D shell electrodes. In a single cycle, we put the 3D shell MEA back into acetone from water to flatten it, then we put it back into water to fold it up. This reversible folding and unfolding offers the potential for reuse of the MEAs. Scale bar: 250 μm.

FIGS. 9A-9D show biocompatibility of the shell MEA according to an embodiment of the current invention. Live/dead assay of (A-B) free-floating and (C-D) brain organoid encapsulated in a shell MEA after 24 hours, the free-floating brain organoid A) Calcein AM (green; live); B) Ethidium homodimer-1 (red; dead); the brain organoid encapsulated C) Calcein AM (green; live); within shell MEA, D) Ethidium homodimer-1 (red; dead); within shell MEA. Results indicate biocompatibility of the shell MEA. Scale bar: 500 μm.

FIGS. 10A-10F show 3D shell MEA recordings from encapsulated brain organoids according to an embodiment of the current invention. (A) Image of a quartz wafer-integrated 3D shell MEA. (B) Electrode distribution of the 3D shell MEA around the brain organoid. (C) Typical field potentials recorded from a brain organoid encapsulated within a 3D shell MEA. (D) Representative raster plot of the spontaneous firing of the brain organoid. (E) Representative overlaid spike waveform from channel 1. (F) Comparison of spike distribution from the recorded brain organoid before and after glutamate treatment.

FIG. 11 shows recordings of spontaneous activities of brain organoids measured by the 3D shell MEAs according to an embodiment of the current invention. The three recordings are from three different organoids.

FIGS. 12A-12F show 3D shell MEA recordings from brain organoids in 6-channel (two on each leaflet) shell electrodes according to an embodiment of the current invention. (A) Optical image of the 6-channel 3D shell MEAs. Scale bar: 200 μm. (B) Optical image of a 6-channel 3D shell MEAs encapsulating a brain organoid. (C) Field potential recorded from the 6-channel 3D shell MEAs. (D) Representative raster plot of the recording. (E) Field potential recorded from 6-channel 3D shell MEAs over approximately 10 minutes. (F) Overlaid spike waveform of each channel.

FIGS. 13A-13D provide an example of CAD mask drawings showing the dimensions of the shell electrodes according to an embodiment of the current invention. Design with (A) three electrodes, (B) six electrodes, (C) nine electrodes, (D) 2D vs 3D. The numbers shown are in micrometers.

FIGS. 14A-14C show 3D MEA shell recordings with glutamate stimulation according to an embodiment of the current invention. (A) 3D MEA shell recordings from a brain organoid before and after glutamate stimulation. (B-C) Inter-spike-interval (ISI) density change (B) before and (C) after glutamate stimulation. The new crest emerged around-0.5 indicating the glutamate-related spikes had a longer ISI.

FIGS. 15A-15D show brain organoid recordings using a conventional 2D Maestro MEA system (Axion Biosystem, Atlanta). (A) An optical image of organoids on a commercial MEA plate. The diameter of the well is 10.35 mm. (B) Raster plot of the recording from the MEA plate. (C) Spike waveform of 16 channels from the recording from 2D MEA plate. (D) Zoomed-in spike waveform of a representative channel (4-2).

FIGS. 16A-16F show an example of 2D recording with shell electrodes in pre-folded state according to an embodiment of the current invention. (A) Optical image of the brain organoid placed on the electrodes. Scale bar: 200 μm. (B) Field potential recorded from 2D electrodes. (C) Representative raster plot of the recording. (D) Field potential recorded from 2D electrodes on a larger time scale. (F) Overlaid spike waveform of each channel.

FIGS. 17A-17D shows a comparison of recordings from 2D and 3D electrodes within a shell MEA according to an embodiment of the current invention. (A) Shell MEAs with three electrodes on the leaflets and an additional electrode at the bottom center. (B) Image of an encapsulated organoid within a shell MEA. (C) Stacked spike counts detected from only the bottom electrode as compared to the shell electrodes indicating that that the union of spike events from all over the shell (3D) is greater than that from a single planar 2D electrode. (D) Detailed raster plot of the recordings from the four different electrodes. Scale bar: 500 μm.

FIGS. 18A-18F show an investigation of different electrode configurations according to some embodiments of the current invention. (A) Optical images of the 3D shell electrodes and 2D electrodes in flat (left) and self-folded (right) state. Scale bar: 200 μm. (B) Optical image of the 3D shell electrodes (#1, 2, 3) encapsulating the brain organoid, along with the 2D electrodes (#4, 5, 6, 7). Scale bar: 200 μm. (C) Raster plot of the 3D shell and 2D recordings. (D) Histograms of spike counts having different SNRs, comparing 3D and 2D channels. The graph displays that the 3D channels have a greater capacity to capture signals with different SNRs. (E) The signal-to-noise ratio of the paired spikes recorded by 3D and 2D electrodes. (F) Trend statistics of the 2D channels and 3D channels with glutamate stimulation.

FIG. 19 shows the recording of the spontaneous activities from brain organoids recorded using both 2D and 3D shell electrodes at different distances according to an embodiment of the current invention. No protein or gel coatings were used.

FIGS. 20A-20E show brain organoid recordings over five rounds of glutamate using both 2D and 3D shell electrodes according to an embodiment of the current invention. No protein or gel coatings were used.

FIGS. 21A-21F provide a comparison between experiments results and mechanics simulations for self-folding of the shell MEAs according to an embodiment of the current invention. Comparison between images of experimentally fabricated self-folding SU8 leaflets (top) and FEM simulations (bottom row). Here we show polymers of different thicknesses and top layer exposures have similar curvature shapes. Images (A) and (D) are 8 μm (total thickness) SU8 exposed at 120 mJ/cm². (B) and (E) are 6 μm (total thickness) exposed at 180 mJ/cm², and images (C) and (F) are 4.6 μm exposed at 180 mJ/cm². As the curvature increases, the leaflet tips begin to rest on each other. While our FEM models do not capture this polymer interaction at the leaflet tips, the radius of curvature measurements support the degree of curvature in the laboratory models. Our experiment also assumes that the presence of organoids within the device will prevent full curving of the leaflets, as shown on FIG. 4B.

FIG. 22A-22D show an abaqus self-folding modeling representation for shell MEAs according to an embodiment of the current invention. Abaqus model representation of a 6 μm thick shell whose top layer is exposed to 180 mJ/cm² UV light. (A) Top view with the gold electrodes in yellow, and SU8 polymer shown in blue. (B) Image showing the fully deformed shape after 43 steps. (C) Image showing stress distribution in the overall MEA shell and shows that gold electrodes experience the highest stress. (D) Image representing the stress distributions in the SU8 material alone, showing high stresses at the bottom surface.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

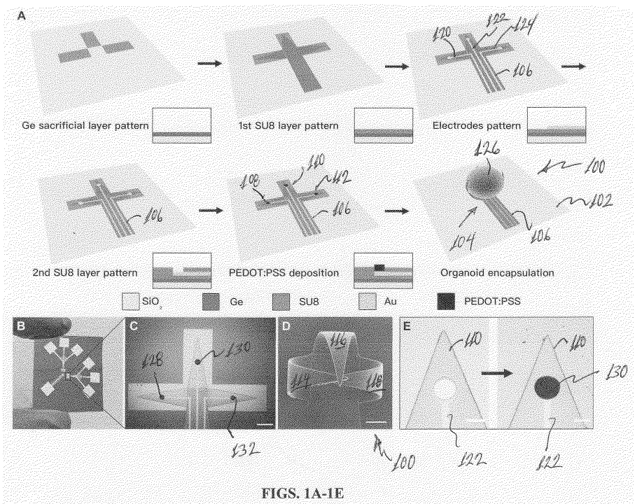

Accordingly, a device 100 for electrically connecting with and holding organoids according to an embodiment of the current invention is shown in FIGS. 1A-1E. The device 100 includes a substrate 102 and a multielectrode array (MEA) 104 attached to the substrate 102. The MEA 104 includes an electrical bus 106 attached to the substrate 102, and a plurality of leaflets (108, 110, 112) each having a first end pivotably connected to the electrical bus 106 while remaining portions thereof (114, 116, 118) are free to move relative to the substrate 102 while remaining connected and pivoting at each first end. In the example of FIGS. 1A-1E, the device 100 has three leaflets (108, 110, 112). However, the general concepts of the current invention are not limited to any particular number of leaflets. There could be one, two, or more than three leaflets in other embodiments.

Each leaflet of the plurality of leaflets (108, 110, 112) includes an electrical wire (120, 122, 124) embedded therein that extends from each respective leaflet into the electrical bus 106. The MEA 104 has a size that is suitable to electrically connect to and hold an organoid 126 with dimensions between 250 μm to 2 cm while at least one leaflet of the plurality of leaflets (108, 110, 112) is pivoted up away from the substrate 102 so as to form a three-dimensional electrical connection with the organoid 126. The 3D size and shape of the MEA 104 can provide the dual effect of securely holding the organoid 126 in place as well as forming electrical connections around portions of the organoid that are out of the plane of the substrate 102. The organoid 126 can include neurons, for example, that can be grown to various sizes up to essentially macroscopic sizes. Such organoids can serve as models for brain function, etc. However, the broad concepts of the current invention are not limited to only neuronal organoids. The organoids can be other cell types that exhibit electrical function, such as, but not limited to, heart tissue.

In an embodiment of the current invention the MEA 104 has a size that is suitable to electrically connect to and hold an organoid 126 that is between 500 μm to 2 mm. In an embodiment of the current invention each said electrical wire (120, 122, 124) includes an electrode (128, 130, 132) attached thereto, the electrode (128, 130, 132) being exposed to provide an electrical connection with the organoid 126. In an embodiment of the current invention each said electrode (128, 130, 132) consists essentially of an electrically conducting polymer material. In an embodiment of the current invention the electrically conducting polymer material is poly-(3,4-ethylenedioxythiophene): poly (styrene sulfonate) (PEDOT: PSS).

The electrodes (128, 130, 132) can be contact pads according to some embodiments of the current invention. In addition, electronic components can be formed such as, but not limited to transistors for example. In other words, electrical wires, contact pads and semiconductor devices, including, but not limited to field effect transistors, can all be included according to some embodiments.

The term “plurality” can mean as few as two up to potentially millions of wires and/or/recording sites on the leaflets.

In an embodiment of the current invention the bus 106 includes a wire that terminates with an electrode attached thereto without entering any of the plurality of leaflets to provide an electrical connection to the organoid substantially coincident with a surface of the substrate. In an embodiment of the current invention at least one of the plurality of leaflets includes a second wire embedded therein, the second wire connecting to a second electrode such that the at least one of the plurality of leaflets connects to at least two different positions of the organoid away from the substrate. In other embodiments, there can be a large number of wires embedded in one or more of the leaflets, for example anywhere from 1-10,000 wires with corresponding electrodes in one or more leaflets. Furthermore, the general concepts of the current invention are not limited to any specific number of leaflets. In various embodiments, there could be one, two, three or more than three leaflets for each MEA, without limitation.

In an embodiment of the current invention the device further includes a plurality of electrodes attached to the substrate at respective separate positions away from said MEA.

In an embodiment of the current invention the plurality of leaflets are responsive to a liquid brought into contact therewith to pivot one of away from or towards the substrate. In an embodiment of the current invention the device further includes a sacrificial layer of material between the substrate and each of the plurality of leaflets so as to hold the plurality of leaflets in a planar configuration until the sacrificial layer of material is removed in order to allow the plurality of leaflets to thereafter be pivotable. In an embodiment of the current invention the sacrificial layer of material is a material that dissolves from coming in contact with a growth medium of the organoid. In an embodiment of the current invention the sacrificial layer of material is one of Ge, Mg, MgO, Fe, FeO, Mo, ZnO or a polymer.

In an embodiment of the current invention the plurality of leaflets each includes a polymer with the wire of each sandwiched therebetween. In an embodiment of the current invention the plurality of leaflets each includes PDMS, a photo-cross-linkable hydrogel, or chemical cross-linkable hydrogel such as, but not limited to, PEGDA, alginate, or gelatin, for example.

In an embodiment of the current invention the plurality of leaflets each comprises first and second layers of photo-responsive material with the wire of each sandwiched therebetween. In an embodiment of the current invention the first and second layers of photo-responsive material have thicknesses and amounts of photo-exposure to correspond to amounts of pivoting. In an embodiment of the current invention the photo-responsive material is SU8.

In an embodiment of the current invention each wire is a wire of at least one of gold, silver, copper, platinum or aluminum.

In an embodiment of the current invention the device further includes a plurality of MEAs attached to the substrate. The general concepts of the current invention are not limited by the number of individual MEA structures on the substrate. There can be one, two, three, or more than three individual MEA structures on the substrate. In some embodiments, there can be at least 10, at least 100 or at least 1,000 of the individual MEA structures on the substrate, for example. In an embodiment of the current invention each MEA of the plurality of MEAs is customized to be electrically connected to and to hold an organoid within a compatible size range. In an embodiment of the current invention the substrate is at least one of a semiconductor substrate or a quartz substrate. In an embodiment of the current invention the substrate is a silicon substrate. In an embodiment, the substrate is a semiconductor material with an oxide layer formed on a surface thereof. For example, the substrate can be, but is not limited to, a silicon-oxide layer on a silicon chip or wafer. However, the general concepts of the current invention are not limited to these examples.

An example of a method of producing a multielectrode array (MEA) for electrically connecting with organoids according to an embodiment of the current invention is shown schematically in FIG. 1A. Although this example is a photolithographic method, the general concepts of the current invention are not limited to only photolithography. The method in this example includes providing a substrate; forming a patterned layer of sacrificial material on the substrate; forming a first polymer layer including a plurality of lower leaflets over corresponding portions of the sacrificial layer of material and a lower bus section in direct contact with the substrate; forming a plurality of wires over the plurality of lower leaflets such that there is at least one wire over each lower leaflet and each of the plurality of wires extends to the lower bus section; and forming a second polymer layer including a plurality of upper leaflets over corresponding lower leaflets and an upper bus section over the lower bus section. Each wire of the plurality of wires is embedded between the first and second polymer layers, and the upper and lower leaflets have a size and a pre-stressed portion such that they fold up to a size to electrically connect with and hold an organoid, the organoid having a maximum dimension of between 250 μm to 2 cm.

According to another embodiment of the current invention, a device for electrically connecting with and holding organoids is produced according to a method according to an embodiment of the current invention.

Some embodiments of the current invention can be useful for brain science studies, toxicity studies, or brain machine interfaces, for example. For example, the MEAs could be used to determine effects of one or more chemicals on the MEAs for variations of the parameters such as, but not limited to compositions, concentrations, etc.

In other embodiments, the MEAs could be part of a computational device, for example.

### Examples

Some further concepts and aspects of the current invention are explained below by with reference to some examples. However, the general concepts of the current invention are not limited to only those examples.

In this section, we report a new silicon wafer-integrated self-folding polymer shell MEA platform for brain organoids according to an embodiment of the current invention. We developed a wafer-scale microfabrication process to create shell MEAs by utilizing a self-folding polymer bilayer (27). The fabrication process is straightforward, making it potentially compatible with MEA plates, tunable, scalable, and cost-effective. The essential element of this embodiment is a self-folding negative photoresist polymer (SU8) bilayer with tunable folding based on the relative thickness and exposure energy. However, the general concepts of the current invention are not limited to only this example. Gold wires and contact pads are integrated within the self-folding bilayer for good insulation with exposed conductive polymer poly-(3,4-ethylenedioxythiophene): poly (styrene sulfonate) (PEDOT: PSS) coated electrodes. The extent of self-folding was guided by a finite element method (FEM) model, with the constitutive relations and volume shrinkage after acetone treatments developed for SU8 crosslinked by different exposure energy. We show that we can create customizable shells for organoids of different sizes, creating a loose or firm contact between the electrodes and the brain organoids. The materials and self-folding processes are biocompatible, and the transparent leaflets of the shell electrodes allow for in-situ bright field, and fluorescence imaging. We demonstrate 3D spatiotemporal recording of brain organoids encapsulated in the 3D shell electrodes with and without glutamate stimulation. We contrast 2D and 3D recordings by comparing cumulative firing and electrical responses to glutamate. The firing was defined via a simple threshold and spike distributions were compared non-parametrically using a Wilcoxon rank sum (permutation) test. The responses to the glutamate intervention were summarized with the non-parametric Mann-Kendall trend test and the inference was again performed by permuting MEAs type (2D, 3D) labels. Both a distributional shift in firing, with higher firing rate and a stronger estimated glutamate mediated trend were detected with 3D MEAs. Together, our platform and studies demonstrate a distinct 3D methodology for mapping electrical activity in brain organoids, offering a larger recording contact area as compared to conventional MEAs.

### Results

### Concept and Fabrication of the Shell MEAs

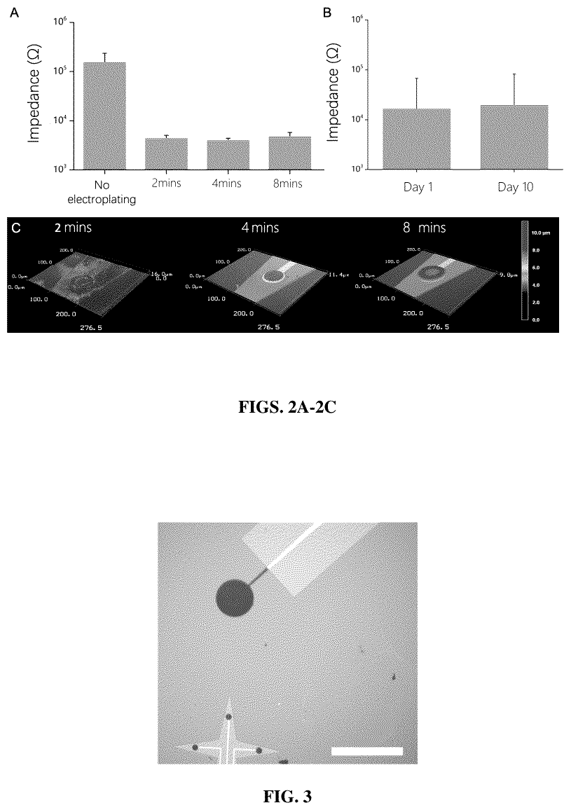

Our development of 3D shell electrodes was inspired by macroscale EEG caps that are used to study the electrical activity of the human brain (28). These caps are typically composed of flexible materials with multiple metal electrodes covering the entire scalp, allowing sampling of electrical signals from all over its 3D shape. Likewise, we designed our shell MEAs to consist of leaflets that can wrap around the surface of the brain organoids. As a proof-of-concept, we patterned three leaflets with three electrodes distributed on the 3D surface. Importantly, our fabrication approach is compatible with alternate designs containing arrays of multiple electrodes and leaflets folding at different angles (27). Briefly, our fabrication process involved the following steps: (a) deposition of sacrificial layer, (b) patterning of first SU8 layer, (c) patterning of gold wiring, (d) patterning of the second SU8 layer, (c) patterning of the PEDOT: PSS electrodes, (f) dissolution of the sacrificial layer and preconditioning, (g) sterilization and placement in cell media, and (h) organoid placement and self-folding as depicted in FIG. 1A. Our current process utilized five photomasks, and we typically fabricated eight shells with three electrodes each on a 3-inch silicon (opaque, SiO2 coated) or quartz (transparent) wafer (FIG. 1B). We chose SU8 for our shell MEAs since it is a popular negative photoresist and has been already widely utilized in microfluidics and micro-electromechanical systems (MEMS) processes (29). We have previously reported the fabrication process and demonstrated the self-folding mechanism of 3D SU8 architectures with solvent exchange (FIGS. 1C and 1D) (27). Due to its widespread use in microsystem fabrication, there is an abundance of technical knowledge on its processing, biocompatibility, and case of integration with other modules that may be integrated in the future for chemical or optical interrogation of organoids (30). We chose gold for the wiring due to its high electrical conductivity and good biocompatibility. We added a conductive polymer PEDOT: PSS coating on the electrode to reduce impedance and minimize the modulus mismatch for the MEAs/organoid contact (FIG. 1E, FIGS. 2A-2C) (19, 31). A reference electrode was placed near the shell electrodes for data acquisition (FIG. 3). Our fabrication and folding process is compatible with AutoCAD mask design and multilayer photolithography and, as a result, scalable.

### Brain Organoid Characterization

We generated the brain organoids used in this study from human induced pluripotent stem cells (NIBSC8 line). We differentiated neuroprogenitor cells (NPCs) (time zero) and then brought a NPC cell suspension to form 3D brain organoids under constant gyratory shaking for up to 8-10 weeks as previously described (8). Mature brain organoids are homogeneous in size ranging from 400 to 600 μm, and consist of neurons, astrocytes, and oligodendrocytes (FIGS. 4A-4C).

In FIGS. 4A and 4B, we show an eight-week brain organoid stained with neuronal markers MAP2, β-III-Tubulin (green), neuroprogenitor marker, Nestin (red), and astrocyte marker, GFAP (red). FIG. 4C shows gene expression of neural markers over time of differentiation, covering stages of NPCs to 10 weeks of differentiation. The maturation over time resulted in an increase in mature neuronal (NeuN), astrocyte (GFAP), and oligodendrocyte (MBP) markers and a decrease in neuroprogenitor markers (Nestin). The presence of mature neurons, astrocytes, and mature oligodendrocytes is key to the brain organoid functionality, which is measured by the system's electrical activity. Maturation of neurons provides increased synaptic formation, while astrocytes support this process. Oligodendrocytes provide myelin sheaths around the axons, which reduce the ion leakage and capacitance of the cell membrane for efficient electrical signal transmission (32, 33). We previously demonstrated that following our protocol we have up to 40% of myelinated axons (8), which is known to be challenging to model in vitro with human brain cells.

### Tunability of Fold Angle, Shape, and Electrode Pattern

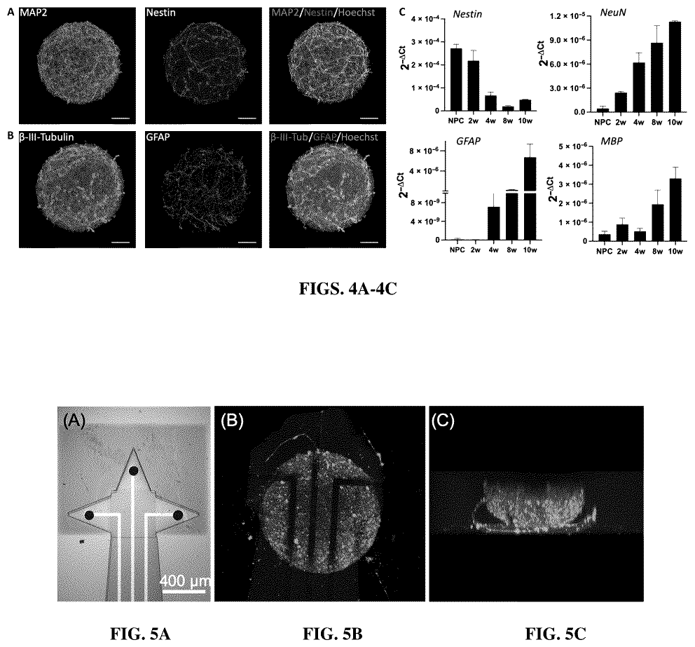

We combined numerical simulation and experimental trials to develop a rational and reliable design for self-folding to enable reproducible assembly and ensure good contact between the organoids and shell MEA electrodes. An optimal shell MEA device should fit the outer contour of the target brain organoids like a cap (FIGS. 5A-5C). The critical parameters that control fold angle include SU8 bilayer thickness and UV exposure.

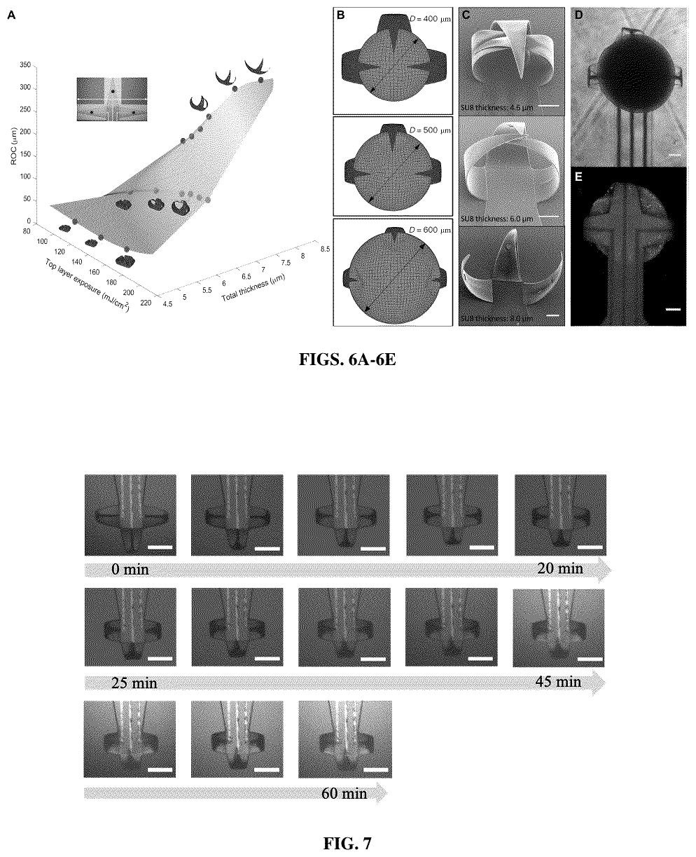

We developed a FEM model and used it to simulate the self-folding behavior of the polymer shell together with the electrodes (Details in the Supplemental Information Notes 1 and 2, below). This model allows us to independently tune the design parameters, including the overall size and geometry, the bilayer/electrode thickness and UV exposure of the top and bottom layers, and predict the fully equilibrated structures after folding in simulations. For typical shell MEAs presented in FIG. 1C, we ran simulations to compare experimental results and study the effects of SU8 bilayer thickness and the exposure energy difference between the two layers on the final folded shell shape (FIG. 6A). Overall, the simulation results clearly demonstrate that the folding increases (i.e. smaller radius of curvature (ROC)) as the thickness decreases. As we expanded the thickness of the SU8 bilayer, the folding decreased due to higher bending stiffness more than the counter-effect caused by the mismatch strain in the two layers. Furthermore, the exposure energy difference between the SU8 bilayer also served an important role in the final desired 3D shape of the shell electrode. As the exposure energy of the top layer increases, the overall folding decreases. The exposure energy difference between the SU8 bilayer generates an overall polymerization gradient across the bilayer. The polymerization gradient causes a mismatch strain along the bilayer thickness and provides a bending moment for the SU8 bilayer to fold spontaneously. We can rationally predict the folding by FEM and in principle produce 3D shell architectures from the 100 μm to 10 cm scale with our lithographic integration process (27). This large range of size tunability is advantageous for recording activities from organoids of different sizes.

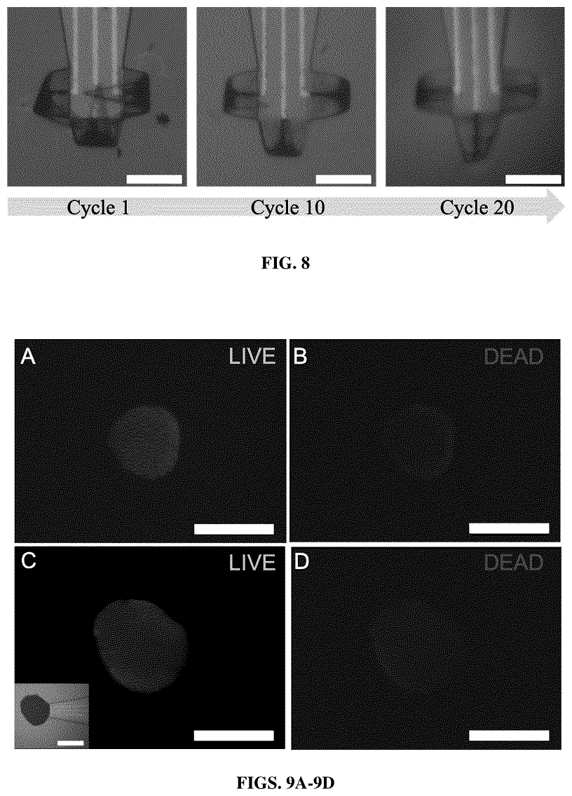

We compared the FEM results with the experimental measurements by using three conditions of the SU8 bilayer with different thickness and top layer UV exposure (from top to bottom: 4.6 μm, 180 mJ/cm² UV exposure; 6.0 μm, 180 mJ/cm² UV exposure; 8.0 μm, 120 mJ/cm² UV exposure), and the bottom layers were fully crosslinked (240 mJ/cm² UV exposure). These different conditions yielded bilayers folding to different extents which agreed with the simulation results (FIG. 6C). Next, we investigated how the shell electrodes captured brain organoids of varied sizes. In general, the brain organoids used here had diameters between 400 μm and 600 μm. Thus, we simulated how the different shell electrodes encapsulated the brain organoids with 400 μm, 500 μm, and 600 μm diameters, respectively (FIG. 6B). These simulation results matched with our experimental observations, where the brain organoids were well encapsulated in the 3D folded shell electrodes (FIG. 6D). A confocal microscope calcium image of a brain organoid enveloped inside the folded 3D shell electrodes is shown in FIG. 6E (34). The transparent SU8 leaflets of the 3D shell electrodes device offer the potential for future optical stimulation. The timescale of the folding process of the 3D shell electrodes (typically complete within an hour) includes adequate time for sterilization as well as encapsulation of the brain organoids (FIG. 7). The folding of the shell electrodes is reversible with the solvent exchange, which showed the potential for reusable electrodes (FIG. 8). A cytotoxicity test was performed on the encapsulated brain organoid and compared with the results of the free-floating organoid (FIG. 9A-9D) to verify the biocompatibility of the process.

### Feasibility of Spontaneous Activity and Glutamate-Induced Electrical Activity Recording

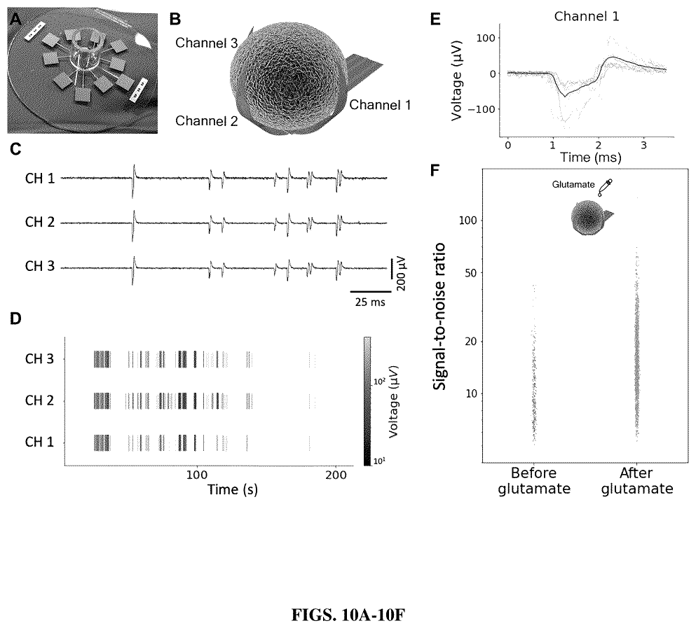

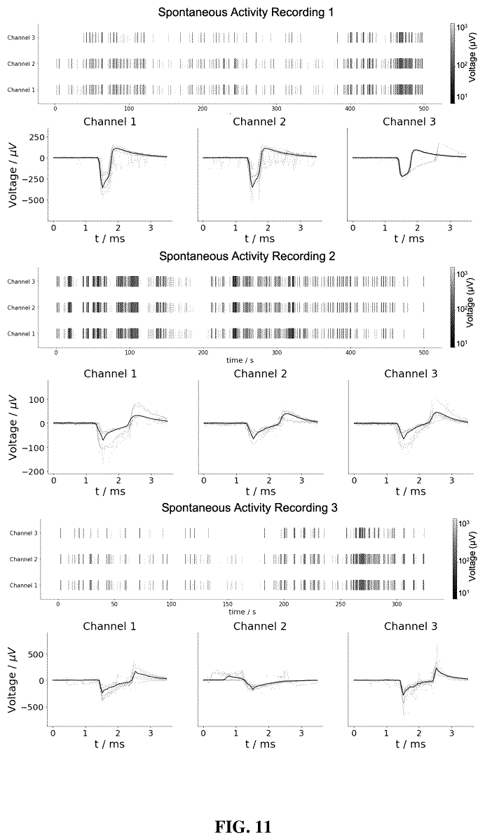

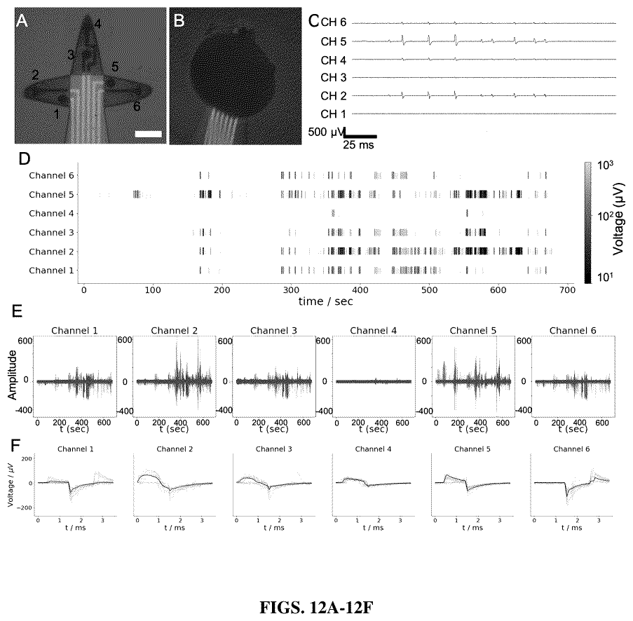

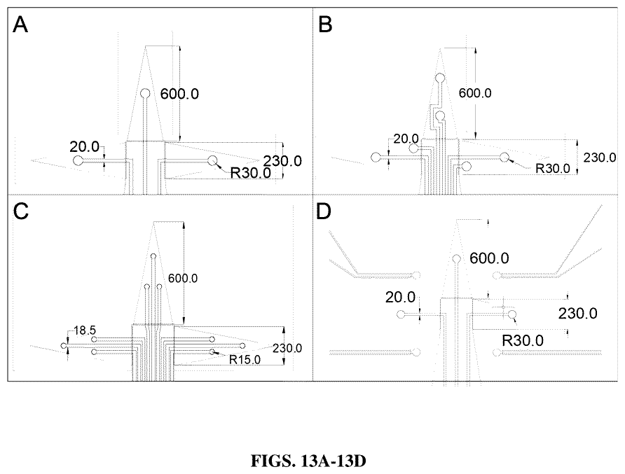

We demonstrate the feasibility of recording 3D spatial electrophysiological activities of the brain organoid across the electrodes on each leaflet. A 9-week brain organoid was placed at the center of the shell electrodes during self-folding, which allowed the organoid to be enveloped within the leaflets with the PEDOT: PSS electrodes in intimate contact with the organoid (FIG. 10B). A glass cylinder was placed around the device to hold the cell media. Electrode outputs were fabricated for signal acquisition (FIG. 10A). The three PEDOT: PSS electrodes on the leaflets successfully recorded the field potential of the enveloped brain organoid (FIG. 10C, FIG. 11). The raster plot (FIG. 10D) shows the recording of the spontaneous activity of the enveloped brain organoid (35, 36). The overlaid spike waveform indicates an average spike duration of ˜2 ms, with varied amplitude during the recording (FIG. 10E). We note that we can add more electrodes to the leaflets to improve the recording (FIGS. 12A-12F, FIGS. 13A-13D).

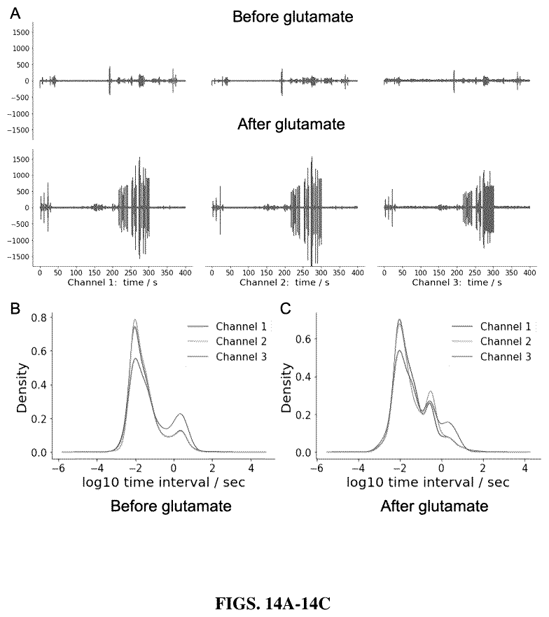

We further confirm that the signal recorded is from the brain organoids by examining the response to 20 μM glutamate neurotransmitter added to the culture media that served as a positive control for the spontaneous recording (23, 37). The spike signal-to-noise ratio (SNR) before and after the glutamate application showed statistically significant amplification (median increased by 57.6%) on the amplitude of firing spikes (FIG. 10F). A detailed clustering also demonstrated that longer inter-spike-interval (ISI) was observed in the glutamate-induced signals (FIGS. 14A-14C).

### Design of Different Electrode Configurations

To gain insight into the relative merits of 3D vs. 2D recording and proximal vs. distal electrodes, we designed an array that features four electrodes on the bottom surface in addition to the 3D shell MEA electrodes. This configuration also demonstrates the case with which our fabrication methodology can be tuned for different electrode combinations.

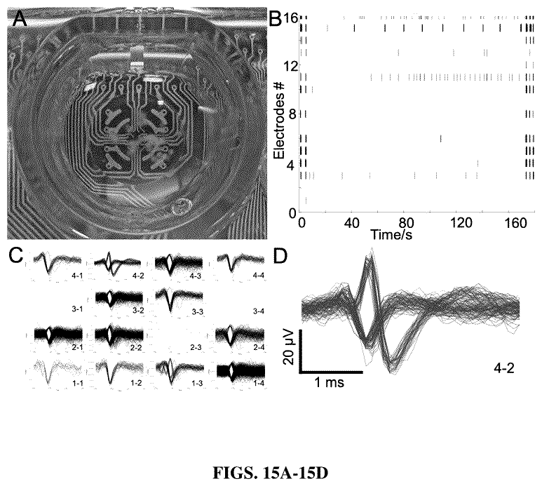

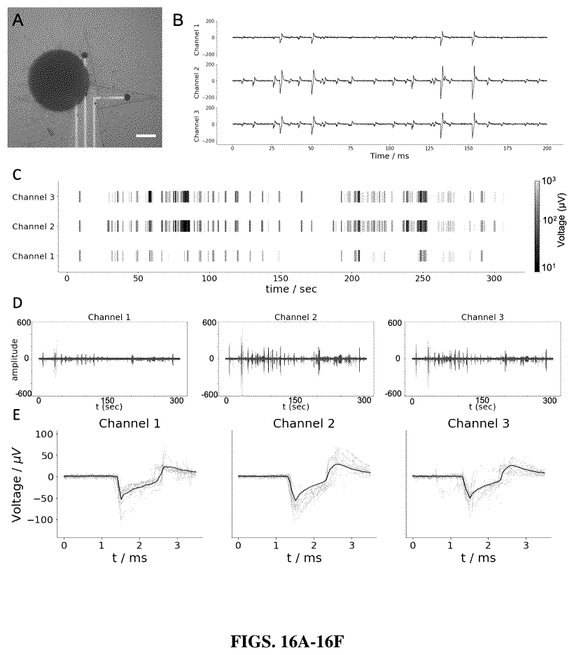

While the conventional MEA recording methodology is well established, the recording from 3D multi-electrode devices is still at a very early stage. In previous studies, researchers have compared 3D recordings with conventional 2D recordings, but these are typically different organoids (24). We also carried out brain organoid recording using a 2D MEAs system (Axion BioSystems, FIGS. 15A-15D) and from our shell MEAs in an unfolded format (FIGS. 16A-16F). Both showed similar spike duration of approximately 2 ms. These studies suggest the feasibility of our recording platform to perform recordings in 2D and allow us to compare these 2D recordings with the 3D recordings.

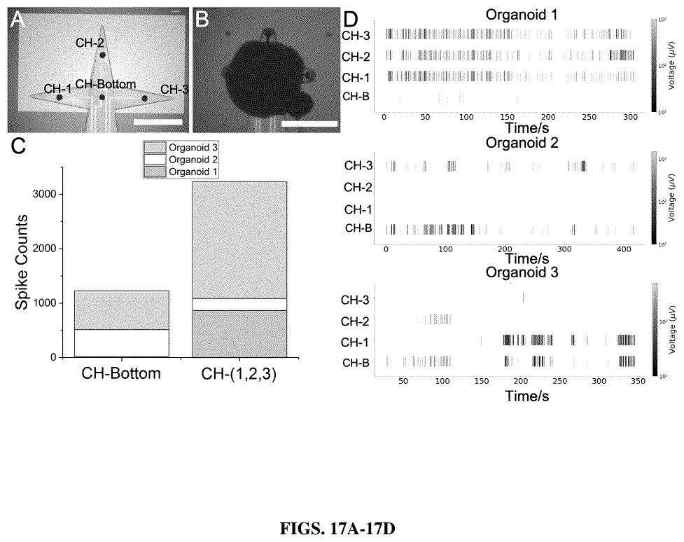

Of note, the 3D MEAs offer more recording electrode pads with greater contact area and spread around the organoid. To directly observe the influence of these features, we designed 3D shell electrodes with three electrodes, one on each leaflet and an additional fourth electrode on the bottom center of the shell (FIG. 17A). Once the shell electrodes were folded and the organoid was encapsulated (FIG. 17B), we were able to record not only from the 3D folded shell electrodes but also from the bottom electrode. We observed that the cumulative spike counts from the three folded electrodes were significantly larger than that from the bottom electrode alone (FIG. 17C). While it is mathematically necessary that the union of spike events is larger than the events from a single electrode, FIGS. 17A-17D demonstrate the scope of the difference. We also observed signal heterogeneity between different electrodes indicating that such MEAs could probe spatiotemporal electrical activity (FIG. 17D). Organoid 1 is the most extreme since most spiking occurred away from the bottom electrode. The stacked bar chart (FIG. 17C) shows that electrodes 1, 2, and 3 bring independent information that is lost if only the bottom electrode is considered.

With our shell electrodes methodology, we can fold distal electrodes into proximal electrodes with 3D spatial distribution. Due to the sample variation across brain organoids, a systematic study on the difference between the recording from 2D (pre-folding) electrodes and 3D (folded) shell electrodes from the organoids is challenging and has been lacking in the published literature. Hence, we designed an electrode system and performed experiments in which we have both the 2D electrodes (#4, 5, 6, 7 in FIG. 18A) and 3D shell electrodes (#1, 2, 3 in FIG. 18A) able to record signals simultaneously from the same brain organoid. This also allowed us to investigate the effect of distance between the electrode and brain organoid. Prior to the folding process, all the electrodes were equidistant to the center of the flat shell (FIG. 18A). Once the MEA shell folded, the leaflets encapsulated the brain organoid so that specific electrodes were proximal to the brain organoid (FIG. 18B) while others were not. Achieving such localization can be difficult for conventional MEAs since the location of the brain organoids seeded on 2D MEAs cannot be well-controlled as they are not encapsulated, and the only proximal electrode location is the exact bottom of the spherical organoid.

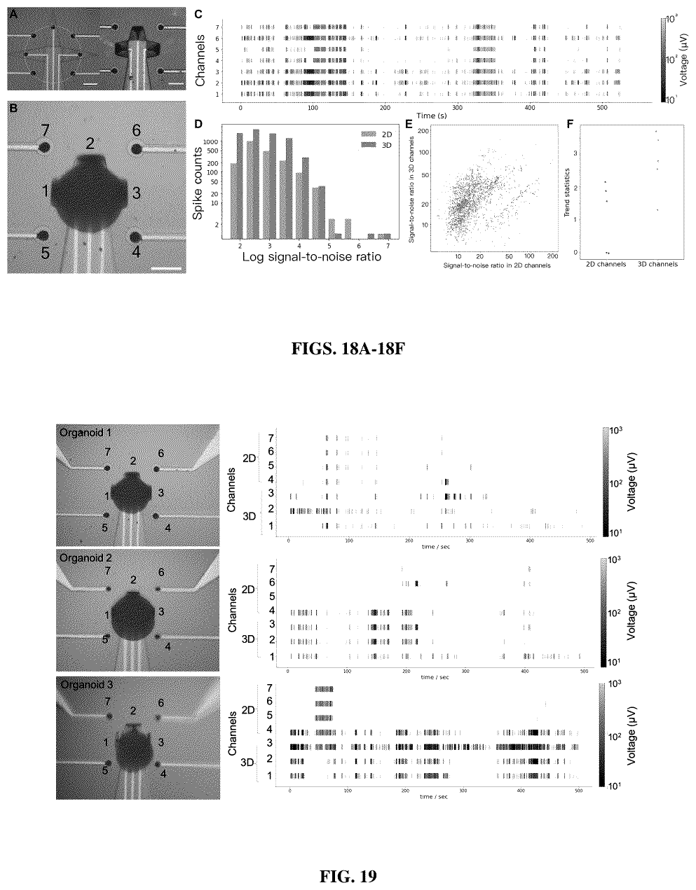

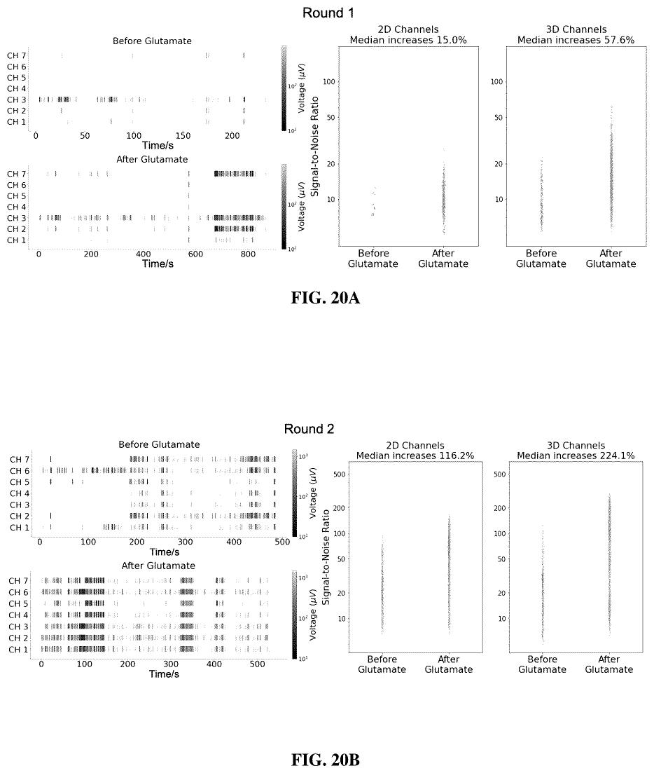

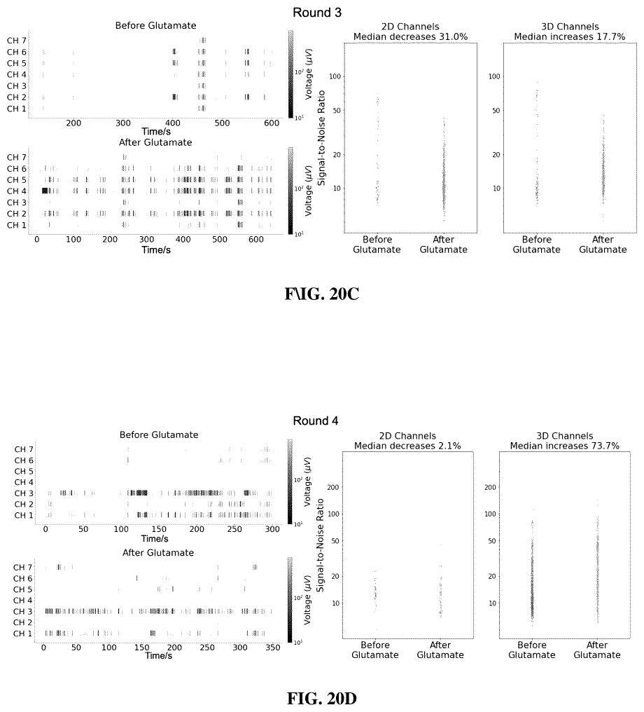

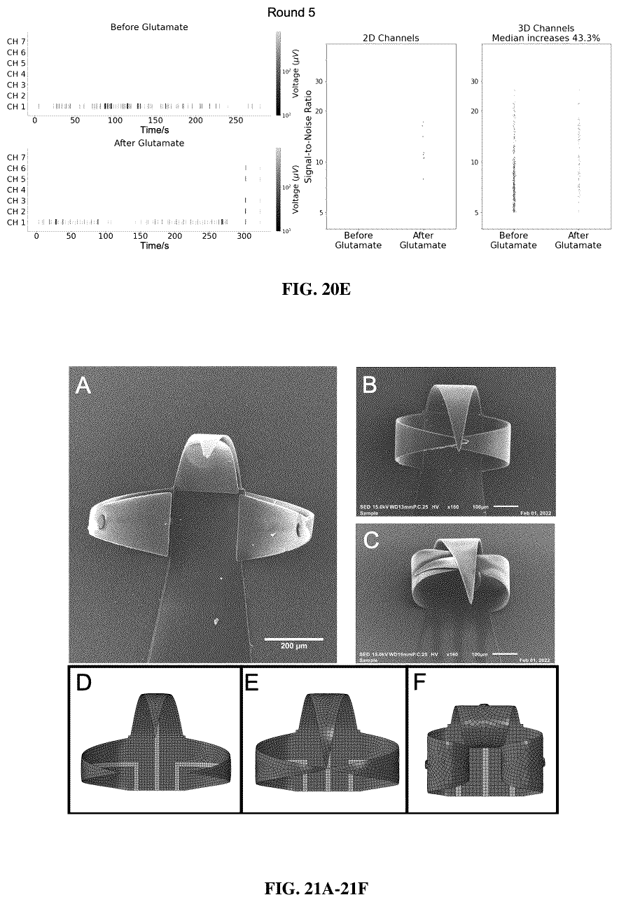

We note that in 2D MEA recordings, a cell adhesive protein or Matrigel® coating is needed to enhance attachment and promote neurite grown from the organoid to electrodes, while no coatings are needed in the shell MEAs. The 3D shell MEA secures the organoid within its grasp and keeps it relatively stable as compared to 2D MEAs during motion with the electrodes proximal to the organoid. Additionally, the absence of a surface coating in 3D MEAs prevents individual cell migration from the organoids and neurite outgrowth, which is unavoidable in 2D MEAs, where a coating is essential to keep organoids attached to the electrodes. Thus, in 2D MEAs, even distant electrodes will be in touch with unknown number of cells and neurites, leading to higher variability in recordings. In our experiments (FIG. 18A), the 2D distance electrodes were not in contact with the cells or neurites. We recorded the field potentials from the four 2D electrodes and three 3D electrodes, as shown in the raster plot (FIG. 18C, FIG. 19). With five rounds of spontaneous recording from 3 different brain organoids, we detected 7,785 spikes from the 3D shell electrodes and 2,025 spikes from the 2D planar electrodes. The results indicate that as the 3D shell electrodes folded up to reach the surface of the 3D organoid, they could detect more spikes than the distanced electrodes from the brain organoid (FIG. 18D). Furthermore, to conduct a direct and fair comparison of the recording quality, we analyzed the 1860 spikes detected both by the 3D shell electrodes and the 2D planar electrodes. The signal-to-noise ratio (SNR) of the same spikes in the 3D shell electrodes channels was significantly higher (yielding a 42% increase in the median, p<0.005) than in the 2D channels (FIG. 18E). We also compared the sensitivity between 3D and 2D recording in response to glutamate stimulations (FIGS. 20A-20E). We stimulated the brain organoid for five rounds with 20 μM glutamate, and the 3D shell electrodes detected a significantly stronger increase in electrophysiological activities (quantified by Mann-Kendall's z of spike amplitudes, p=0.02) (FIG. 18F) (38). In summation, for brain organoids, the recording from 3D shell electrodes detected more spikes and were more sensitive to stimulation-induced activities, thus augmenting the electrophysiological recordings compared to the original 2D electrodes with a potential for spatial analysis in the future.

### DISCUSSION

In summary, we have reported a wafer-integrated self-foldable 3D shell MEA neural interface for brain organoids. We have created both a rational design and customizable fit for organoids of different sizes, which is important for recording from organoids during different stages of maturation and development. The inclusion of polymer (SU8 and conductive PEDOT: PSS coatings) as compared to significantly more rigid silicon or metallic materials minimizes modulus mismatch between the recording device and the organoid (39). It is noteworthy that our differentially cross-linked bilayer self-folding approach could also be utilized with even softer polymers and hydrogels in future iterations (40). Our self-folding process is highly parallel, compatible with conventional photolithography, and does not require any probes or transfer steps. Hence, it can be implemented at wafer-scale for facile microintegration with alternate microelectronic, microfluidic, and micromechanical components. We can also readily vary the spacing and layout of recording electrodes and wires using conventional lithographic processes, which is necessary to increase inputs/outputs (I/O). MEAs can be used for stimulation in addition to recording. Furthermore, our integration approach is amenable to the integration of complementary metal-oxide-semiconductor (CMOS) or related components. We have demonstrated robust 3D recording from our 3D shell electrodes and evaluated their performance relative to 2D electrodes and in the presence of stimulants.

In the current study, we manually inserted the organoids into the shell electrodes. In addition, with integration of microwell or microfluidic interfaces, we anticipate that we can grow the organoids inside the shell electrodes so that we can form high-throughput arrays of electrode-integrated organoids (26, 41). Based on the current design, we can also increase the number of I/O or utilize CMOS sensors for higher recording resolution by utilizing other interconnect layouts and higher resolution lithography, create porous leaflets for enhanced oxygen and nutrient transportation, and stable long-term recording and stimulation. Also, it is conceivable that foldable electrodes with surface patterns or protrusions can be used so that they penetrate and facilitate recording from within the organoid and these strategies are compatible with our microfabrication and self-folding approach (22).

This study opens avenues for more in-depth analysis of the brain organoid connectome of neural cells and brain organoid to machine interfaces. AI-based analysis methods of human EEGs (42, 43) lend themselves for the analysis of such recordings. Ultimately, such analysis might allow the realization of primitive cognitive functions in brain organoids.

### Materials and Methods

### Fabrication and Actuation of 3D Shell Electrodes

First, we patterned a 50 nm thick germanium (Ge) sacrificial layer on either a silicon dioxide (SiO₂) or quartz (transparent) wafer. Then, we spin coated a layer of SU8 2002 or 2005 (Kayaku, Westborough, MA) on top of the substrate, and we patterned and fully crosslinked the first layer of SU8 via photolithography through a photomask and developed in SU8 developer for 1 minute to define the shape of the first layer. On top of the first SU8 layer, we spin-coated a ˜2.7 μm Shipley SC 1827 photoresist (Kayaku, Westborough, MA). We defined patterns of electrodes using photomasks followed by 1-minute development in 351 Developer (1:5) (Kayaku, Westborough, MA). We deposited the electrodes (Cr 10 nm adhesion promoter, Au 50 nm) using thermal evaporation and lift-off. We patterned the bilayer of SU8 on top of the electrode layer, partially crosslinked, thus forming a SU8 solvent-responsive bilayer and electrically insulating the electrodes. We used SU8 2005 at 3000 rpm for both SU8 layers to get a bilayer with a thickness of 8.0 μm; we used a SU8 mixture (25% SU8 2005, 75% SU8 2002) for both SU8 layers to get a bilayer with a thickness of 6.0 μm; we used a SU8 mixture (50% SU8 2005, 50% SU8 2002) for both SU8 layers to get a bilayer with a thickness of 4.6 μm.

### Electroplating of the Conductive PEDOT: PSS Electrode Coating

We prepared the PEDOT: PSS electrolyte by mixing 10 mM EDOT (Sigma Aldrich, St. Louis, MO) and PSS (Sigma Aldrich, St. Louis, MO) 0.4 wt % in DI water. After cleaning the wafer with acetone and oxygen plasma, we connected the electrode pads with copper wires using alligator clips. We then attached the working electrode (Pt) to the anode and the sample to the cathode for electroplating. We set the current density at 12.5 mA/cm² and deposited the PEDOT: PSS for 4 min to create a 10 μm thick coating. After electroplating, we rinsed the sample with DI water, and a dark blue PEDOT: PSS coating can be seen on top of the Au electrode layer.

### Characterization of PEDOT: PSS Coatings

The impedance of PEDOT: PSS coatings was measured using an Intan RHD recording system (Intan Technologies, Los Angeles, CA) in 1× Phosphate Buffered Saline (PBS) at 1000 Hz. The height profile was acquired using a Keyence laser scanning microscope VK-X100.

### Actuation of 3D Shell Electrodes

We dissolved the Ge sacrificial layer in 5% hydrogen peroxide. Once the Ge sacrificial layer disappeared, we immersed the flat shell electrodes in acetone for 5 minutes to pre-condition (remove any uncrosslinked SU8) the folding of the shell electrodes. We subsequently placed the 3D shell electrodes back in the water and rinsed them three times. We actuated the self-folding of the shell MEAs by placing back in aqueous solutions.

### Brain Organoids

We differentiated brain organoids from induced Pluripotent Stem Cell (iPSC) NIBSC-8 cell line (UK National Institute for Biological Standards and Control (NIBSC)), following our in-house two-step protocol (8). The NIBSC-8 iPSC cell line is mycoplasma-free, with a normal female karyotype. Briefly, we differentiated iPSCs in a monolayer to neuroprogenitor cells (NPCs) using serum-free, chemically defined neural induction medium (Gibco, Thermo Fisher Scientific). We expanded the NPCs and a single cell suspension was distributed into uncoated 6-well plates and cultured under constant gyratory shaking (80 rpm, 19 mm orbit) to form 3D aggregates. After 48 hours, we induced differentiation with serum-free, chemically defined differentiation medium (Neurobasal electro medium (Gibco, Thermo Fisher Scientific) supplemented with 1×B27-electro (Gibco, Thermo Fisher Scientific), 2× glutamax, 10 ng/ml GDNF (Gemini), and 10 ng/ml BDNF (Gemini) and 5% PenStrep). We differentiated brain organoids for 8 to 10 weeks prior to recording. By this time, the brain organoids consist of different types of neurons, astrocytes, and oligodendrocytes (8).

### Immunohistochemistry

We fixed the brain organoids at 8 weeks of differentiation with 2% PFA for 45 minutes, blocked with 10% goat serum, 1% BSA, 0.15% saponin in 1×PBS for 1 hour and stained with primary antibodies diluted in blocking solution for 48 hours. After three washes with 1% BSA/0.15% saponin in 1×PBS, we stained the organoids with secondary antibodies for 24 hours, stained them with Hoechst for one hour, washed twice, and mounted them on the glass slide with Immu-Mount. We used the following antibodies: mouse anti-Map2 (Clone AP-20, Sigma Aldrich), mouse anti β-III-Tubulin (Clone SDL.3D10, Sigma Aldrich), rabbit anti-GFAP (policolonal, Dako), rabbit anti-Nestin (policolonal, Sigma Aldrich), Alexa-Fluor488 goat-anti-mouse and Alexa-Flour 568 goat-anti-rabbit IgGs.

### RNA Extraction and RT-PCR

We extracted RNA from the NPCs as well as brain organoids at 2, 4, 8, and 10 weeks of differentiation using Quick RNA extraction kit (Zymo). We quantified the integrity of RNA with NanoDrop. We reverse transcribed cDNA using M-MLV Reverse Transcriptase (Promega) and random hexamer primers as described previously (44). We used the TaqMan gene expression assay to perform Real-Time PCR.

### Encapsulation of Brain Organoids in the 3D Shell Electrodes

As the shell electrodes folded up, we sterilized the glass chamber and 3D shell electrodes with 70% ethanol. We rinsed the device with 1×PBS and then cell media. Later, we added the organoid into the chamber with a pipette and used a pipette tip to gently move the organoid into the 3D shell electrodes from the non-leaflet direction.

### Calcium Imaging

For calcium imaging, first we fabricated the 3D shell electrodes on a transparent quartz wafer. After organoid encapsulation, we applied 0.4× fluo-4 direct calcium reagent (Sigma Aldrich, St. Louis, MO) into the organoid media and incubated it at 37° C. for 1 hour, and then let it rest at room temperature for 15 min before imaging. We imaged the sample with a Nikon Al confocal microscope (Nikon, Tokyo, Japan).

### Cytotoxicity Test

We used a LIVE/DEAD viability/cytotoxicity kit for mammalian cell (Molecular Probes, Eugene, OR). We added 20 μL of 1 mM Calcein AM and 20 μL of 2 mM Ethidium homodimer-1 in 10 mL PBS solution. We added the chemicals to brain organoids for 10 mins, then rinsed with PBS and cell media before imaging with a Nikon AZ100 microscope (Nikon, Tokyo, Japan).

### Scanning Electron Microscope (SEM) Imaging

We sputter-coated gold on the 3D shell electrodes for 1 minute. We took SEM images using a JEOL SEM (JSM IT100).

### Finite Element Method (FEM) Simulation

We used finite element method (FEM) with Abaqus® to simulate the folding of SU8 bilayers. We modeled each layer of the SU8 bilayer and the gold electrode as homogenous, isotropic material. The mechanical properties of each layer of the SU8 bilayer were defined by its Young's modulus (E), Poisson's ratio (=0.3), and pre-existing strain (c) before folding. Additional details of the model are in the Supplementary Information.

### 2D Recording and 3D Recording Process

For the 2D recordings with flat (pre-folded) SU8 bilayer shells, we first coated the 2D MEAs with Matrigel® solution and incubated it for 1 hour. We then replaced the Matrigel® solution with cell media and placed the brain organoid on the electrodes. Before starting the recording, we put the device and the organoid back into the incubator for approximately 24 hours for neurite outgrowth and environment stabilization.

For 2D recordings with the commercial Axion system, the MEA plate was coated with poly-L-Ornithine 15 μg/mL and laminin 10 μg/mL.

For the 3D recordings, after actuation and sterilization of the shell electrodes, we loaded the cell media and placed the organoid onto the semi-folded shell electrodes. The shell kept folding to firmly encapsulate the brain organoid. We put the device and the organoid back into the incubator for 24 hours or longer time for environment stabilization before we started the recording. No protein or gel coatings were used for 3D shell MEA recordings.

### Recording Data Acquisition

For the electrophysiology recording, we connected the outputs of the shell electrodes to the printed circuit board (PCB) interface using micro alligator clips. We connected the Omnetics connector on the PCB to a 32-channel headstage, transferring the electrophysiology recording to an RHD recording controller. The sampling rate of the recording was 20 kHz. We acquired all recordings in a grounded Faraday cage on a vibration isolation table.

### Signal Processing

After preprocessing the raw signals, we detected spikes separate for each channel using a threshold-based method, where the threshold is automatically set to be 5σₙ. We estimated the noise level σ robustly using the formula σₙ=median (|Sₜ|/0.6745), where |Sₜ| is the absolute signal amplitudes. After spike locations were detected, we isolated them by windows of length 3.5 ms. We then regarded locations of the largest amplitudes as event times.

### Spike Merging

For the comparison of 3D and 2D channels, we merged the spikes so that one spike train represented the 3D recordings and one for the 2D recordings. To accomplish this, we detected and merged identical spikes by multiple electrodes to avoid double counts. This was done by treating spikes with peak times differing less than 2 ms (one waveform length) as the same spike, and hence summarizing them as one single spike with maximum normalized amplitude (45, 46).

### Spike Comparison Between 3D and 2D Recordings

After merging spikes into one sequence for 3D electrodes and one sequence for 2D electrodes, we detected 7,785 spikes from the 3D electrodes and 2,025 spikes from 2D electrodes, among these 1,860 spikes were detected in common by both electrodes. We calculated the normalized spike amplitudes by dividing the spike amplitudes divided by noise level, on, (henceforth SNR).

We also compared the sensitivity of 3D and 2D recording in response to stimulation. We stimulated the organoid with glutamate, yielding five rounds of recording data. Since glutamate is known to increase neural activity (37), we investigated whether 3D or 2D electrodes are more sensitive to glutamate mediated changes. We quantified the trend via the Mann-Kendall's z statistics of spike amplitudes (38). This statistic is larger for increasing trends and is normalized to be comparable across different recordings. We used the permutation test, permuting the 2D versus 3D labels, for statistical inference.

### REFERENCES

1. T. Hartung, Thoughts on limitations of animal models. Parkinsonism Relat. Disord. 14 Suppl 2, S81-3 (2008).
2. E. Di Lullo, A. R. Kriegstein, The use of brain organoids to investigate neural development and disease. Nat. Rev. Neurosci. 18, 573-584 (2017).
3. C. A. Trujillo, R. Gao, P. D. Negraes, J. Gu, J. Buchanan, S. Preissl, A. Wang, W. Wu, G. G. Haddad, I. A. Chaim, A. Domissy, M. Vandenberghe, A. Devor, G. W. Yeo, B. Voytek, A. R. Muotri, Complex Oscillatory Waves Emerging from Cortical Organoids Model Early Human Brain Network Development. Cell Stem Cell. 25, 558-569.e7 (2019).
4. U. Marx, T. Akabane, T. B. Andersson, E. Baker, M. Beilmann, S. Beken, S. Brendler-Schwaab, M. Cirit, R. David, E. M. Dehne, I. Durieux, L. Ewart, S. C. Fitzpatrick, O. Frey, F. Fuchs, L. G. Griffith, G. A. Hamilton, T. Hartung, J. Hoeng, H. Hogberg,. A. Roth, Biology-inspired microphysiological systems to advance patient benefit and animal welfare in drug development. ALTEX. 37 (3), 365-394. (2020).
5. A. Roth, MPS-WS Berlin 2019, Human microphysiological systems for drug development. Science. 373 (6561), 1304-1306 (2021).
6. M. A. Lancaster, M. Renner, C.-A. Martin, D. Wenzel, L. S. Bicknell, M. E. Hurles, T. Homfray, J. M. Penninger, A. P. Jackson, J. A. Knoblich, Cerebral organoids model human brain development and microcephaly. Nature. 501, 373-379 (2013).
7. A. M. Paşca, S. A. Sloan, L. E. Clarke, Y. Tian, C. D. Makinson, N. Huber, C. H. Kim, J.-Y. Park, N. A. O'Rourke, K. D. Nguyen, S. J. Smith, J. R. Huguenard, D. H. Geschwind, B. A. Barres, S. P. Paşca, Functional cortical neurons and astrocytes from human pluripotent stem cells in 3D culture. Nat. Methods. 12, 671-678 (2015).
8. D. Pamies, P. Barreras, K. Block, G. Makri, A. Kumar, D. Wiersma, L. Smirnova, C. Zang, J. Bressler, K. M. Christian, G. Harris, G.-L. Ming, C. J. Berlinicke, K. Kyro, H. Song, C. A. Pardo, T. Hartung, H. T. Hogberg, A human brain microphysiological system derived from induced pluripotent stem cells to study neurological diseases and toxicity. ALTEX. 34, 362-376 (2017).
9. B. Cakir, Y. Xiang, Y. Tanaka, M. H. Kural, M. Parent, Y.-J. Kang, K. Chapeton, B. Patterson, Y. Yuan, C.-S. He, M. S. B. Raredon, J. Dengelegi, K.-Y. Kim, P. Sun, M. Zhong, S. Lee, P. Patra, F. Hyder, L. E. Niklason, S.-H. Lee, Y.-S. Yoon, I.-H. Park, Engineering of human brain organoids with a functional vascular-like system. Nat. Methods. 16, 1169-1175 (2019).
10. I. Chiaradia, M. A. Lancaster, Brain organoids for the study of human neurobiology at the interface of in vitro and in vivo. Nat. Neurosci. 23, 1496-1508 (2020).
11. W. A. Anderson, A. Bosak, H. T. Hogberg, T. Hartung, M. J. Moore, Advances in 3D neuronal microphysiological systems: towards a functional nervous system on a chip. In Vitro Cell. Dev. Biol.-Anim. 57 (2), 191-206 (2021).
12. C. A. Thomas Jr, P. A. Springer, G. E. Loeb, Y. Berwald-Netter, L. M. Okun, A miniature microelectrode array to monitor the bioelectric activity of cultured cells. Exp. Cell Res. 74, 61-66 (1972).
13. J. Pine, Recording action potentials from cultured neurons with extracellular microcircuit electrodes. J. Neurosci. Methods. 2, 19-31 (1980).
14. M. E. Spira, A. Hai, Multi-electrode array technologies for neuroscience and cardiology. Nat. Nanotechnol. 8, 83-94 (2013).
15. M. Durens, J. Nestor, M. Williams, K. Herold, R. F. Niescier, J. W. Lunden, A. W. Phillips, Y.-C. Lin, D. M. Dykxhoorn, M. W. Nestor, High-throughput screening of human induced pluripotent stem cell-derived brain organoids. J. Neurosci. Methods. 335, 108627 (2020).
16. A. Hai, A. Dormann, J. Shappir, S. Yitzchaik, C. Bartic, G. Borghs, J. P. M. Langedijk, M. E. Spira, Spine-shaped gold protrusions improve the adherence and electrical coupling of neurons with the surface of micro-electronic devices. J. R. Soc. Interface. 6, 1153-1165 (2009).
17. J. T. Robinson, M. Jorgolli, A. K. Shalek, M.-H. Yoon, R. S. Gertner, H. Park, Vertical nanowire electrode arrays as a scalable platform for intracellular interfacing to neuronal circuits. Nat. Nanotechnol. 7, 180-184 (2012).
18. J. Abbott, T. Ye, L. Qin, M. Jorgolli, R. S. Gertner, D. Ham, H. Park, CMOS nanoelectrode array for all-electrical intracellular electrophysiological imaging. Nat. Nanotechnol. 12, 460-466 (2017).
19. Y. Liu, A. F. McGuire, H.-Y. Lou, T. L. Li, J. B.-H. Tok, B. Cui, Z. Bao, Soft conductive micropillar electrode arrays for biologically relevant electrophysiological recording. Proc. Natl. Acad. Sci. 115, 11718-11723 (2018).
20. J. Cools, Q. Jin, E. Yoon, D. Alba Burbano, Z. Luo, D. Cuypers, G. Callewaert, D. Braeken, D. H. Gracias, A Micropatterned multielectrode shell for 3D spatiotemporal recording from live cells. Adv. Sci. 5, 1700731 (2018).
21. A. Kalmykov, C. Huang, J. Bliley, D. Shiwarski, J. Tashman, A. Abdullah, S. K. Rastogi, S. Shukla, E. Mataev, A. W. Feinberg, K. J. Hsia, T. Cohen-Karni, Organ-on-e-chip: Three-dimensional self-rolled biosensor array for electrical interrogations of human electrogenic spheroids. Sci. Adv. 5, eaax0729 (2019).
22. D. A. Soscia, D. Lam, A. C. Tooker, H. A. Enright, M. Triplett, P. Karande, S. K. G. Peters, A. P. Sales, E. K. Wheeler, N. O. Fischer, A flexible 3-dimensional microelectrode array for in vitro brain models. Lab Chip. 20, 901-911 (2020).
23. A. Kalmykov, J. W. Reddy, E. Bedoyan, Y. Wang, R. Garg, S. K. Rastogi, D. Cohen-Karni, M. Chamanzar, T. Cohen-Karni, Bioelectrical interfaces with cortical spheroids in three-dimensions. J. Neural Eng. 18 (2021).
24 Y. Park, C. K. Franz, H. Ryu, H. Luan, K. Y. Cotton, J. U. Kim, T. S. Chung, S. Zhao, A. Vazquez-Guardado, D. S. Yang, K. Li, R. Avila, J. K. Phillips, M. J. Quezada, H. Jang, S. S. Kwak, S. M. Won, K. Kwon, H. Jeong, A. J. Bandodkar, M. Han, H. Zhao, G. R. Osher, H. Wang, K. Lee, Y. Zhang, Y. Huang, J. D. Finan, J. A. Rogers, Three-dimensional, multifunctional neural interfaces for cortical spheroids and engineered assembloids. Sci. Adv. 7 (2021).
25. Y. Park, T. S. Chung, J. A. Rogers, Three dimensional bioelectronic interfaces to small-scale biological systems. Curr. Opin. Biotechnol. 72, 1-7 (2021).
26. P. Le Floch, Q. Li, Z. Lin, S. Zhao, R. Liu, K. Tasnim, H. Jiang, J. Liu, Stretchable Mesh Nanoelectronics for 3D Single-Cell Chronic Electrophysiology from Developing Brain Organoids. Adv. Mater. e2106829 (2022).
27 Q. Huang, T. Deng, W. Xu, C. K. Yoon, Z. Qin, Y. Lin, T. Li, Y. Yang, M. Shen, S. M. Thon, J. B. Khurgin, D. H. Gracias, Solvent Responsive Self-Folding of 3D Photosensitive Graphene Architectures. Adv. Intel. Sys. 2000195 (2020).
28 C.-T. Lin, L.-W. Ko, M.-H. Chang, J.-R. Duann, J.-Y. Chen, T.-P. Su, T.-P. Jung, Review of wireless and wearable electroencephalogram systems and brain-computer interfaces—a mini-review. Gerontology. 56, 112-119 (2010).
29. K. V. Nemani, K. L. Moodie, J. B. Brennick, A. Su, B. Gimi, In vitro and in vivo evaluation of SU8 biocompatibility. Mater. Sci. Eng. C 33, 4453-4459 (2013).
30. A.-N. Cho, Y. Jin, Y. An, J. Kim, Y. S. Choi, J. S. Lee, J. Kim, W.-Y. Choi, D.-J. Koo, W. Yu, G.-E. Chang, D.-Y. Kim, S.-H. Jo, J. Kim, S.-Y. Kim, Y.-G. Kim, J. Y. Kim, N. Choi, E. Cheong, Y.-J. Kim, H. S. Je, H.-C. Kang, S.-W. Cho, Microfluidic device with brain extracellular matrix promotes structural and functional maturation of human brain organoids. Nat. Commun. 12, 4730 (2021).
31. J. P. Neto, P. Baião, G. Lopes, J. Frazão, J. Nogueira, E. Fortunato, P. Barquinha, A. R. Kampff, Does Impedance matter when recording spikes with polytrodes? Front. Neurosci. 12, 715 (2018).
32. M. Chesnut, T. Hartung, H. Hogberg, D. Pamies, Human oligodendrocytes and myelin in vitro to evaluate developmental neurotoxicity. Int. J. Mol. Sci. 22 (15), 7929 (2021).
33. M. Chesnut, H. Paschoud, C. Repond, L. Smirnova, T. Hartung, M. G. Zurich, H. T. Hogberg, D. Pamies, Human ipsc-derived model to study myelin disruption. Int. J. Mol. Sci. 22 (17), 9473 (2021).
34 H. Renner, M. Grabos, K. J. Becker, T. E. Kagermeier, J. Wu, M. Otto, S. Peischard, D. Zeuschner, Y. TsyTsyura, P. Disse, J. Klingauf, S. A. Leidel, G. Seebohm, H. R. Schöler, J. M. Bruder, A fully automated high-throughput workflow for 3D-based chemical screening in human midbrain organoids. Elife. 9 (2020).
35. H. G. Rey, C. Pedreira, R. Quian Quiroga, Past, present and future of spike sorting techniques. Brain. Res. Bull. 119, 106-117 (2015).
36. M. S. Lewicki, A review of methods for spike sorting: the detection and classification of neural action potentials. Network. 9, R53-78 (1998).
37. Y. Han, H. Zhu, Y. Zhao, Y. Lang, H. Sun, J. Han, L. Wang, C. Wang, J. Zhou, The effect of acute glutamate treatment on the functional connectivity and network topology of cortical cultures. Med. Eng. Phys. 71, 91-97 (2019).
38. A. I. McLeod, Kendall rank correlation and Mann-Kendall trend test. R Package Kendall (2005) (available at http://ftp.unibayreuth.de/math/statlib/R/CRAN/doc/packages/Kendall.pdf).
39. S. P. Lacour, G. Courtine, J. Guck, Materials and technologies for soft implantable neuroprostheses. Nat. Rev. Mater. 1, 16063 (2016)
40. O. Erol, A. Pantula, W. Liu, D. H. Gracias, Transformer hydrogels: A review. Adv. Mater. Technol. 4, 1900043 (2019).
41 N. Brandenberg, S. Hoehnel, F. Kuttler, K. Homicsko, C. Ceroni, T. Ringel, N. Gjorevski, G. Schwank, G. Coukos, G. Turcatti, M. P. Lutolf, High-throughput automated organoid culture via stem-cell aggregation in microcavity arrays. Nat. Biomed. Eng. 4, 863-874 (2020).
42. M. Antonijevic, M. Zivkovic, S. Arsic, A. Jevremovic, Using AI-based classification techniques to process EEG data collected during the visual short-term memory assessment. J. Sensors. (2020).
43. M. Golmohammadi, A. H. Harati Nejad Torbati, S. Lopez de Diego, I. Obeid, J. Picone, Automatic analysis of EEGs using big data and hybrid deep learning architectures. Front. Hum. Neurosci. 13, 76 (2019).
44 S. Modafferi, X. Zhong, A. Kleensang, Y. Murata, F. Fagiani, D. Pamies, H. T. Hogberg, V. Calabrese, H. Lachman, T. Hartung, L. Smirnova, Gene-environment interactions in developmental neurotoxicity: A case study of synergy between chlorpyrifos and CHD8 knockout in human BrainSpheres. Environ. Health Perspect. 129, 77001 (2021).
45. K. Chen, Y. Jiang, Z. Wu, N. Zheng, H. Wang, H. Hong, HTsort: Enabling Fast and Accurate Spike Sorting on Multi-Electrode Arrays. Front. Comput. Neurosci. 15, 657151 (2021).
46. C. Rossant, S. N. Kadir, D. F. M. Goodman, J. Schulman, M. L. D. Hunter, A. B. Saleem, A. Grosmark, M. Belluscio, G. H. Denfield, A. S. Ecker, A. S. Tolias, S. Solomon, G. Buzsaki, M. Carandini, K. D. Harris, Spike sorting for large, dense electrode arrays. Nat. Neurosci. 19, 634-641 (2016).
47. S. Yang, J. Qu, Computing thermomechanical properties of crosslinked epoxy by molecular dynamic simulations Polymer 53, 4806-4817 (2012).
48. A. Bandyopadhyay, P. K. Valavala, T. C. Clancy, K. E. Wise, G. M. Odegard, Molecular modeling of crosslinked epoxy polymers: The effect of crosslink density on thermomechanical properties, Polymer 52, 2445-2452 (2011).
49. N. R. Langley, K. E. Polmanteer, Relation of elastic modulus to crosslink and entanglement concentrations in rubber networks, J. Polym. Sci. Polym. Phys. Ed. 12, 1023 (1974).

The Young's modulus as a function of the energy of exposure amount. The data was experimentally measured.

TABLE S1
Modeling parameters used

| Exposure | Young's modulus E ± | Young's modulus E ± |
|---|---|---|
| energy I | STDEV (GPa): | STDEV (GPa): FEM as |
| (mJ/cm²) | Experiment | given by Eq. 1 |
| 56.75 | 0 | 0.0129 |
| 120 | 4.99 ± 0.38 | 4.99 |
| 196 | 5.95 ± 0.04 | 5.99 |
| 240 | 6.13 ± 0.07 | 6.10 |

The temperature gradient as a function of bilayer shell thickness and varying top layer energy exposures, and average radius of curvature of the folded leaflets.

TABLE S2
Modeling parameters used

| Total | Top layer UV | | Average |
|---|---|---|---|
| thickness of | exposure I_{Top} | Temperature | radius of |
| SU8 bilayer (μm) | (mJ/cm²) | gradient Dₑₚₛ | curvature (μm) |
| 4.6 | 120 | −25.81 | 47.33 |
| | 150 | −17.28 | 46.57 |
| | 180 | −13.62 | 54.40 |
| 6.0 | 120 | −11.63 | 70.80 |
| | 150 | −7.79 | 108.02 |
| | 180 | −6.14 | 135.16 |
| 8.0 | 120 | −4.91 | 177.70 |
| | 150 | −3.29 | 274.59 |
| | 180 | −2.59 | 328.53 |

### Supplemental Note S1: Mechanical Modeling of SU8 in Different Cross-Linking States

We used the finite element method (FEM) to simulate the self-folding of the SU8 bilayers. We modeled each layer of the SU8 leaflet and the gold electrode as a homogenous, isotropic material. The mechanical properties of each layer of the SU8 bilayer are defined by its Young's modulus (E), Poisson's ratio (v=0.3) and pre-existing strain (ε) before folding.

To model the Young's modulus of SU8 as a function of the UV exposure, we set up a series of experiments to measure the material stiffness as a function of the exposure energy for a 5 μm film. Each energy level was repeated nine times, and we measured the Young's modulus of the film after curing. Since it is challenging to measure the critical energy level that starts to cure the material, we considered 56.75 mJ/cm², as the critical energy obtained from the previous analysis, as the energy level to transition SU8 from a liquid to solid state and thus corresponds to zero Young's modulus, as summarized in Table S1 which gives the experimental value of Young's modulus of the SU8 film after the specified energy amount of UV exposure.

By using the values in table S1, we obtained the exponential decay function for the sample stiffness as a function of the exposure energy. We identified the Young's modulus as a function of exposure energy as,

$$\\ \begin{matrix}E ( k ) = {E}_{0} - Δ E \cdot exp ( - k / {k}_{0} ) & ( 1 )\end{matrix}$$

where E₀=6.15 GPa is the limiting Young's modulus with enough exposure, ΔE=27.26 GPa and k₀=38.06 mJ/cm² are two constants obtained through fitting and determine the shape of the curve. Eq. (1) provides the basic function for the Young's modulus of the top/bottom SU8 layers in the experiments.

We considered the effect of the solvent exchange on the top/bottom SU8 layers by tuning the pre-existing strain & in each layer of the bilayer to reflect the volume change after being rinsed in acetone and put into water. Several reports in the molecular dynamics (MD) literature have discussed the volume/density of epoxy-based polymer materials after crosslinking. However, there are contradicting results as some show that a higher crosslink ratio causes the material to shrink while others show that the material swells. However, these volume changes are on the order of only a few percent [47-48] and are not large enough to explain the large-scale deformation of the experimental samples in our current study. Here, we considered that the volume change that happens to the top/bottom layers is caused by dissolving of uncrosslinked SU8 monomers in acetone, leaving voids that cause the material to shrink in water because of the highly hydrophobic features of SU8. According to the theory of rubber-like elasticity, the crosslink density n can be calculated from the Young's modulus E and the absolute temperature T as,

$$\\ \begin{matrix}n = \frac{E}{3 RT} & ( 2 )\end{matrix}$$

where R is the gas constant. It is clear that n & E and thus we have the ratio of crosslinked SU8 monomers given by,

$$\\ \begin{matrix}c = \frac{n}{{n}_{0}} = \frac{E ( k )}{{E}_{0}} & ( 3 )\end{matrix}$$

where n₀ is the fully crosslinked SU8 corresponding to a Young's modulus of E₀. Based on the above analysis, the final volume V_{end} of the material after dissolving in acetone and shrinkage in water is given by the initial volume V₀ as V_{end}=AcV₀, where A is an unknown factor and A=1 simply means all uncrosslinked SU8 gets dissolved in acetone. Hence, the pre-existing strain of the equilibrium SU8 bilayer before folding, in all the directions, is given by,

$$\\ \begin{matrix}ε = \sqrt[{3}_{}]{Ac} - 1 , & ( 4 )\end{matrix}$$

We note that there is only one unknown parameter A for our material model given by Eqs. (1) and (4). We estimated its value by testing different A's and comparing them to the experimental results for a specific leaflet curvature. We identified A=0.87 as yielding a radius of curvature that gives the closest results when compared to our experimentally obtained models. All the simulation results reported in the main text are obtained by using this constant value.

### Supplemental Note S2: Finite Element Modeling of the SU8 Bilayer Under Thermal Strains

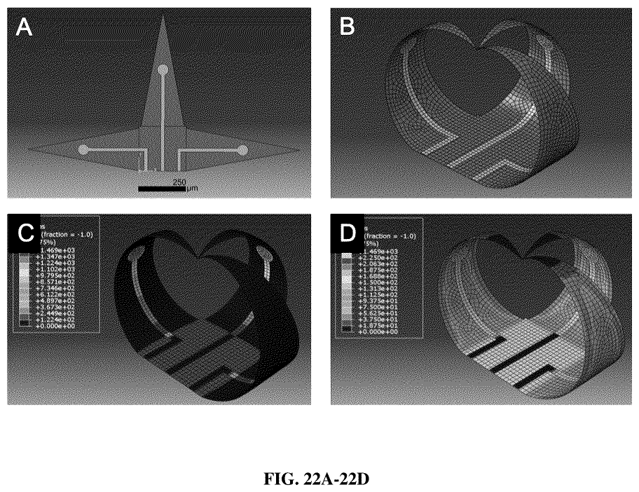

We developed a FEM model in Abaqus to predict the 3D folded structure of a bilayer SU8 leaflet from a 2D pattern design. We modeled the geometry of the bilayer leaflets (2D pattern, thickness), the Young's modulus, Poisson's ratio and pre-existing strain with Abaqus CAE and solved for the fully equilibrated folded structure by using the Standard/Implicit solver on our local workstation (Table S2). To make the simulation efficient, we modeled the initial pattern of the SU8 bilayers by a 3D planar shell with the thickness defined by the actual polymer bilayer (1) and enough integration points (11) along the thickness to account for the heterogeneous normal strain distribution, which provides the driving force for folding. To apply the mismatch strain to the top and bottom layer (as given by the different pre-strain εₜₒₚ and ε_{bottom}), we prescribed a predefined temperature field by directly assigning the temperature (T) gradient, dT/dt through the bilayer thickness, which is given by,

$$\\ \begin{matrix}\frac{dT}{dt} = \frac{{ε}_{top}}{{C}_{1} α {t}^{{}_{} {C}_{2}}} & ( 5 )\end{matrix}$$

where εₜₒₚ gives the mismatch strain of the top and bottom layer (we assume the strain in the bottom layer is zero as it is always fully cross-linked by exposing to UV of =240 mJ/cm²) in the FEM bilayer model as given Eq. (4), a can be any positive value for the thermal expansion coefficient as defined for the material property (we use 0.001/° C. here), and t is the thickness of the bilayer. We included two numerical parameters C₁ and C₂ here, because the mismatch strain occurs only at the interface between two bonded layers in experiment, instead of a continuous gradience of strain as what is used in FEM. We observed in experiments that the conformation of the 3D folded structure was much more sensitive to the change of t of a small value than t of a large value. We systematically varied the C₁ and C₂ values and compared the simulations with the experimental observations for bilayers with different thicknesses and UV to identify that C₁=0.35 and C₂=3 yield the best match (FIG. 21A-21F).

We modeled the gold (Au) electrode layer by another shell with a thickness of 85 nm and material properties given by (E=79 GPa, v=0.415). We assembled this layer on top of the SU8 bilayer surface, as shown in FIG. 22A-22D.

We also rationalized the relationship between the radius of curvature as a function of UV exposure intensity by post-processing the deformed Abaqus/Standard models. We performed further processing of the deformed Abaqus/Standard models in the computer-aided-design (CAD) software SOLIDWORKS, where we visualized the radius of curvature of the leaflets. Our analysis showed a positive correlation between the radius of curvature, shell thickness, and top layer UV exposure amount.

We exported a raw object mesh file (OBJ) consisting of around 6,000 triangles from Abaqus into the SOLIDWORKS software for this analysis. OBJ is a simple file format that represents the 3D geometry alone. We chose SOLIDWORKS to analyze the model due to the ease and availability of tools that can accurately describe the radius of curvature along different parts of a curved surface. To capture the radius of curvature, we identified a set of points along the centerline of any of the three leaflets. In our simulations, we assumed all three folded leaflets had the same curvature and fold shape. We chose the slicing tool along on a geometric plane parallel to the curved leaflet to generate a two-dimensional sketch of points. We converted the approximately 130 points formed using this “Slice” into a spline line, with a tolerance matching the thickness of each model. It is important to note that the curvature along the leaflets is not a perfect circle but elliptical in shape. To accurately describe the radius of curvature, we examined the bottom, middle, and top thirds of each leaflet, where the SOLIDWORKS's spline line feature was able to generate a minimum radius curvature of a circle that could fit the chosen set of points.

In total, we measured four radii at the bottom, middle, top, and whole leaflet, then averaged these together to generate a numerical description of the degree of folding.

Some aspects of the current invention are directed to the following:

1. The device wherein the sacrificial layer of material is one of Ge, Mg, MgO, Fe, FeO, Mo, ZnO or a polymer.

2. The device wherein the plurality of leaflets each comprises a polymer with the wire of each sandwiched therebetween.

3. The device wherein the plurality of leaflets each comprises PDMS, a photocrosslinkable hydrogel, or chemical crosslinkable hydrogel.

4. The device wherein the plurality of leaflets each comprises first and second layers of photo-responsive material with the wire of each sandwiched therebetween.

5. The device wherein the first and second layers of photo-responsive material have thicknesses and amounts of photo-exposure to correspond to amounts of pivoting.

6. The device wherein the photo-responsive material is SU8.

7. The device wherein each wire is a wire of at least one of gold, silver, copper, platinum or aluminum.

8. The device further comprising a plurality of MEAs attached to the substrate.

9. The device wherein each MEA of the plurality of MEAs is customized to be electrically connected to and to hold an organoid within a compatible size range.

10. The device wherein the substrate is at least one of a dielectric, semiconductor substrate or a quartz substrate.

11. The device wherein the substrate is the dielectric coated semiconductor substrate, the semiconductor substrate being a silicon substrate.

12. The method further comprising a plurality of electrodes attached to the substrate at respective separate positions away from said MEA.

13. The method further comprising a sacrificial layer of material between the substrate and each of the plurality of leaflets so as to hold the plurality of leaflets in a planar configuration until the sacrificial layer of material is removed in order to allow the plurality of leaflets to thereafter be pivotable.

14. The method wherein the sacrificial layer of material is a material that dissolves on coming into contact with a growth medium of the organoid.

15. The method wherein the sacrificial layer of material is one of Ge, Mg, MgO, Fe, FeO, Mo, ZnO or a polymer.

16. The method wherein the plurality of leaflets each comprises a polymer with the wire of each sandwiched therebetween.

17. The method wherein the plurality of leaflets each comprises PDMS, a photocrosslinkable hydrogel, or chemical crosslinkable hydrogel.

18. The method wherein the plurality of leaflets each comprises first and second layers of photo-responsive material with the wire of each sandwiched therebetween.

19. The method wherein the first and second layers of photo-responsive material have thicknesses and amounts of photo-exposure to correspond to amounts of pivoting.

20. The method wherein the photo-responsive material is SU8.

21 The method wherein each wire is a wire of at least one of gold, silver, copper, platinum or aluminum.

22. The method further comprising a plurality of MEAs attached to the substrate.

23 The method wherein each MEA of the plurality of MEAs is customized to be electrically connected to and to hold an organoid within a compatible size range.

24. The method wherein the substrate is at least one of a semiconductor substrate or a quartz substrate.

25. The method wherein the substrate is the semiconductor substrate, the semiconductor substrate being a silicon substrate.

While various embodiments of the present invention are described above, it should be understood that they are presented by way of example only, and not limitation. Thus, the breadth and scope of the present invention should not be limited by any of the described illustrative embodiments but should instead be defined only in accordance with the following claims and their equivalents.

The embodiments illustrated and discussed in this specification are intended only to teach those skilled in the art how to make and use the invention. In describing embodiments of the disclosure, specific terminology is employed for the sake of clarity. However, the disclosure is not intended to be limited to the specific terminology so selected. The above-described embodiments of the disclosure may be modified or varied, without departing from the invention, as appreciated by those skilled in the art in light of the teachings herein. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

We claim:

1. A device for electrically connecting with and holding organoids, comprising:
a substrate; and
a multielectrode array (MEA) attached to said substrate,
wherein said MEA comprises:
an electrical bus attached to said substrate, and
a plurality of leaflets each having a first end pivotably connected to said electrical bus while remaining portions thereof are free to move relative to said substrate while remaining connected and pivoting at each said first end,
wherein each leaflet of said plurality of leaflets comprises an electrical wire embedded therein that extends from each respective leaflet into said electrical bus,
wherein said MEA has a size that is suitable to electrically connect to and hold an organoid that is between 250 μm to 2 cm while at least one leaflet of said plurality of leaflets is pivoted up away from said substrate so as to securely hold the organoid in place and form a three-dimensional electrical connection with said organoid.

2. The device according to claim 1, wherein said MEA has a size that is suitable to electrically connect to and hold an organoid that is between 500 μm to 2 mm.

3. The device according to claim 1, wherein each said electrical wire comprises an electrode attached thereto, said electrode being exposed to provide an electrical connection with said organoid.

4. The device according to claim 3, wherein each said electrode consists essentially of an electrically conducting polymer material, semiconductor, metallic or hybrid interface.

5. The device according to claim 4, wherein said electrically conducting polymer material is poly-(3,4-ethylenedioxythiophene): poly (styrene sulfonate) (PEDOT: PSS).

6. The device according to claim 1, wherein said bus comprises a wire that terminates with an electrode attached thereto without entering any of said plurality of leaflets to provide an electrical connection to said organoid substantially coincident with a surface of said substrate.

7. The device according to claim 1, wherein at least one of said plurality of leaflets comprises a second wire embedded therein, said second wire connecting to a second electrode such that said at least one of said plurality of leaflets connects to at least two different positions of said organoid away from said substrate.

8. The device according to claim 1, further comprising a plurality of electrodes attached to said substrate at respective separate positions away from said MEA.

9. The device according to claim 1, wherein said plurality of leaflets are responsive to a liquid brought into contact therewith to pivot one of away from or towards said substrate.

10. The device according to claim 1, further comprising a sacrificial layer of material between said substrate and each of said plurality of leaflets so as to hold said plurality of leaflets in a planar configuration until said sacrificial layer of material is removed in order to allow said plurality of leaflets to thereafter be pivotable.

11. The device according to claim 10, wherein said sacrificial layer of material is a material that dissolves from coming in contact with a growth medium of said organoid.

12. A method of producing a multielectrode array (MEA) for electrically connecting with organoids, comprising:
providing a substrate;
forming a pattern layer of sacrificial material on said substrate;
forming a first polymer layer comprising a plurality of lower leaflets over corresponding portions of said sacrificial layer of material and a lower bus section in direct contact with said substrate;
forming a plurality of wires over said plurality of lower leaflets such that there is at least one wire over each lower leaflet and each of said plurality of wires extends to said lower bus section; and
forming a second polymer layer comprising a plurality of upper leaflets over corresponding said lower leaflets and an upper bus section over said lower bus section,
wherein said plurality of wires are embedded between said first and said second polymer layers, and
wherein said upper and lower leaflets have a size and a pre-stressed portion such that they fold up to a size to electrically connect with and hold an organoid, said organoid having a maximum dimension of between 250 μm to 2 cm.

13. The method according to claim 12, wherein said MEA has a size that is suitable to electrically connect to and hold an organoid that is between 500 μm to 2 mm.

14. The method according to claim 12, wherein each said electrical wire comprises an electrode attached thereto, said electrode being exposed to provide an electrical connection with said organoid.

15. The method according to claim 14, wherein each said electrode consists essentially of an electrically conducting polymer material.

16. The method according to claim 15, wherein said electrically conducting polymer material is poly-(3,4-ethylenedioxythiophene): poly (styrene sulfonate) (PEDOT: PSS).

17. The method according to claim 12, wherein said bus comprises a wire that terminates with an electrode attached thereto without entering any of said plurality of leaflets to provide an electrical connection to said organoid substantially coincident with a surface of said substrate.

18. The method according to claim 12, wherein at least one of said plurality of leaflets comprises a second wire embedded therein, said second wire connecting to a second electrode such that said at least one of said plurality of leaflets connects to at least two different positions of said organoid away from said substrate.

19. The method according to claim 12, wherein said plurality of leaflets are responsive to a liquid brought into contact therewith to pivot one of away from or towards said substrate.

20. A device for electrically connecting with and holding organoids produced according to the method of claim 12.
